(19) United States
(12) Patent Application Publication
(10) Pub. No.: US 2011/0097737 A1
(43) Pub. Date: Apr. 28, 2011

(54) Methods and Compositions for Targeting Proteins of Interest to the Host Cell Envelope

(75) Inventors: James C. Samuelson, Newburyport, MA (US); Jianying Luo, Ipswich, MA (US)

(73) Assignee: NEW ENGLAND BIOLABS, INC., Ipswich, MA (US)

(21) Appl. No.: 12/673,906

(22) PCT Filed: Aug. 14, 2008
(86) PCT No.: PCT/US08/73126

§ 371 Date: Feb. 17, 2010

Related U.S. Application Data
(60) Provisional application No. 60/965,649, filed on Aug. 21, 2007.
(60) Provisional application No. 60/965,729, filed on Aug. 22, 2007.

(51) Int. Cl. G01N 33/573; C07H 21/04; C07K 14/00; C12N 9/96; C12P 21/06; G01N 33/53; C12N 9/16; C12N 15/63; C12N 5/10

(52) U.S. Cl. 435/7.4; 536/23.4; 530/350; 435/188; 435/69.7; 435/7.1; 435/196; 435/320.1; 435/325

(57) ABSTRACT

Methods and compositions are provided for producing membrane proteins or toxic proteins from recombinant DNA introduced into a prokaryotic host cell by targeting the expressed proteins to the envelope of the host cell. The methods and compositions utilize a protein vehicle fused to a protein of interest. The fusion protein may contain one or more protease cleavage sites to separate the protein of interest from the protein vehicle either in vivo or in vitro. The protein vehicle is characterized by a membrane-tar peptide and a trans-membrane segment separated by a cytoplasmic amino acid sequence that includes a cytoplasmic affinity-binding domain.

### BACKGROUND OF THE INVENTION

Attempts have been made to express recombinant eukaryotic membrane proteins in bacteria but these methods are problematic for reasons that include toxicity and insolubility, and the undesirable formation of inclusion bodies (Grisshammer et al. Biochem. J. 295:571-576 (1993); Tucker and Grisshammer Biochem. J. 317:891-899 (1996); Weiss and Grisshammer Eur. J. Biochem. 269:82-92 (2002); Yeliseev et al. Protein Sci. 14:2638-53 (2005); Krepkiy et al. Protein Expr Purif. 49:60-70 (2006); Yeliseev et al. Protein Expr Purif. 53:153-163 (2007)). In addition there is a lack of reliable methods for overexpression of recombinant membrane proteins in E. coli.

Eukaryotic membrane proteins are targeted to various compartments within a eukaryotic cell by means of an address that is encoded in an N-terminal signal sequence or C-terminal targeting sequence (Emanuelsson et al. Nature Protocols 2:953-971 (2007)). The signal sequence is typically removed during biogenesis.

In bacteria that are not membrane-compartmentalized, most newly translated membrane proteins, even while associated with the ribosome, are recognized by signal recognition particles (SRP) and the SRP receptor protein for targeting to the inner membrane surrounding the bacterial cell. Most inner membrane proteins studied in E. coli use the SRP pathway and the Sec translocase for targeting to the membrane and membrane assembly. In addition, a subset of membrane proteins has an absolute requirement for YidC, a polytopic inner membrane protein of E. coli that aids the membrane insertion process. YidC is essential for E. coli cell viability (Samuelson et al. Nature 406:637-641 (2000); Urbanus et al. EMBO Rep. 2:524-529 (2001); van Bloois et al. J. Biol. Chem. 280:12996-13003 (2005)).

A method of choice for stable expression of proteins that rely on disulfide bonds for their native structure is to export the newly synthesized protein into the E. coli periplasm which contains disulfide bond catalyzing enzymes (SRP-dependent DsbA, DsbB, DsbC and DsbD) (Bardwell, et al. Cell 67:581 (1991); Kamitani et al. EMBO J. 11:57 (1992); Novagen (now EMD Chemicals, Inc., Gibbstown, N.J.) pET system manual (11^{th} ed. January 2007); Missiakas et al. EMBO J. 13:2013-2020 (1994); and Zapun et al. Biochemistry 34:5075-5089 (1995)).

Candidate proteins for export to the periplasm can be identified by an N-terminal signal peptide using signal peptide search algorithms such as Phobius (http://phobius.sbc.su.se/) (Kall et al., Nucleic Acids Res. 35:W429-32 (2007)) and Signal P http://www.cbs.dtu.dk/services/SignalP/) (Emanuelsson et al. Nat Protoc. 2(4):953-71 (2007)).

Export to the periplasm is however limited by the through-put capacity of the Sec protein export machinery (Pérez-Pérez, et al. Biotechnology 12(2):178-80 (1994)) in which the Sec translocase may become saturated and result in the cytoplasmic accumulation of native secreted proteins as well as the over-expressed protein of interest (Wagner et al. Mol. Cell Proteomics 6(9):1527-50 (2007)). This is more commonly observed when the protein of interest is non-native to E. coli and/or is not normally secreted. Alternatively, a non-native protein may become incorrectly folded during synthesis and hence become unsuited for Sec-mediated export across the inner membrane.

### SUMMARY

An embodiment of the invention provides a recombinant DNA for introduction into a prokaryotic host cell having a cytoplasmic compartment and an envelope. The recombinant DNA encodes an N-terminal protein vehicle for transporting a protein of interest fused to the vehicle from the cytoplasmic compartment to the envelope where the encoded protein vehicle includes a membrane targeting peptide, a cytoplasmic affinity-binding domain, and a trans-membrane segment. The DNA encoding the protein vehicle is preferably fused to DNA encoding a protein of interest.

In one embodiment, the encoded membrane-targeting peptide is characterized by a Goldman-Engelman-Steitz (GES) hydrophobicity score of at least 1.52 for a window size of 21. In an additional embodiment, the encoded membrane-targeting peptide and the encoded trans-membrane segment which may be different from each other, nonetheless have an amino acid window of 21 amino acids such that within the 21 amino acid window there is a hydrophobic core sequence of at least 9 amino acids lacking Asp, Glu, Arg, and Lys. In an additional embodiment, the membrane-targeting peptide is YidC-dependent. Examples of YidC-dependent peptides are PVIII, Pf3 coat, and subunit C variant L31F. In an additional embodiment, the 21 amino acid window of the trans-membrane segment is flanked on the N-terminal end by an amino acid sequence that comprises at least 9 amino acids having an overall charge of at least +1. Additionally, the trans-membrane segment may be flanked on the C-terminal end by an amino acid linker sequence which may contain a signal peptidase cleavage site. The amino acid linker sequence may contain a heterologous protease cleavage site. The encoded trans-membrane segment may be TM2 of signal peptidase.

In an embodiment of the invention, the cytoplasmic affinity-binding domain is a carbohydrate-binding domain, for example a chitin-binding domain or a His tag and a strep tag.

In an embodiment of the invention, a fusion protein is provided that includes a membrane-targeting peptide, a cytoplasmic affinity-binding domain, and a trans-membrane segment. The fusion protein may additionally include a protein of interest. For example, the protein of interest may be membrane protein or a toxic protein such as a recombinant restriction endonuclease such as Sau3AI or an isoschizomer thereof.

In an embodiment of the invention, a method is provided for producing a recombinant protein in a gram-negative bacterial host cell that includes obtaining a recombinant DNA encoding a protein vehicle as characterized above fused to a DNA encoding a protein of interest and transforming the host cell with this DNA. The recombinant protein may be purified from the host cell by binding the cytoplasmic affinity-binding domain to an affinity substrate. The affinity substrate may be used to detect, purify and/or assay the amount of the expressed protein of interest. The enzymatic activity of the expressed protein of interest may also be determined. Examples of a protein of interest include Sau3AI and BfuCI. Where the protein of interest contains cysteines but does not require disulfide bridges, then the host cell preferably has a DsbA⁻ phenotype due to a mutation in the dsbA gene.

In an embodiment of the invention, a DNA comprising nucleotide sequence 631-1986 SEQ ID NO:23 is provided.

In an embodiment of the invention, a method is provided for producing a restriction endonuclease in the absence of cognate methylation as described above for Sau3AI or isoschizomer thereof. Additionally a vector is provided that includes the recombinant DNA described above. A host cell is also provided for transforming with the vector.

### BRIEF DESCRIPTION OF THE DRAWINGS

FIG. 1 shows the membrane topology of pVIII-PhoA1 fusion protein with PhoA1 located in the cytoplasm.

FIG. 2 shows the membrane topology of pVIII-TM2 in which the protein of interest is exported to the periplasm. (“TM2” is an abbreviation for any trans-membrane segment.)

FIG. 3 shows the membrane topology of pVIII-8His in which the protein of interest is located between the pVIII and the fusion junction.

FIG. 4 shows an anti-pVIII immunoblot demonstrating expression of various membrane-targeting peptides. pVIII-P2 is a control fusion protein expressed from the parent expression clone pVIII-P2. (“P2” is the abbreviation for the protein of interest.) pVIII-8His contains a cytoplasmic eight-histidine affinity tag. pVIII-CBD contains a cytoplasmic chitin-binding domain (CBD) affinity tag. Protein is not detected without IPTG addition (−) confirming IPTG-inducible expression control with all pMS119-derived plasmids. Lane (M) contains a biotinylated protein ladder (Cell Signaling Technology #77275, Beverly, Mass.) detected by anti-biotin, HRP-linked antibody.

FIG. 5 shows the membrane topology of pVIII-CBD.

FIG. 6 shows the membrane topology of pVIII-CBD-PhoA9 fusion protein.

FIG. 7 shows the membrane topology of pVIII-CBD-Oxa1p fusion protein, where Oxa1p is a eukaryotic membrane protein.

FIG. 8 shows the membrane topology of pVIII-CBD-Oxa8His fusion protein.

FIG. 9 shows the membrane topology of pVIII-CBD-Ek-Oxa8His fusion protein.

FIG. 10 shows the membrane topology of pVIII-CBD-Tev-Oxa8His fusion protein. (“His” is an abbreviation for a histidine peptide for affinity binding. “Tev” is an abbreviation for Tobacco Etch Virus.)

FIG. 11A shows an immunoblot where anti-pVIII monoclonal antibody was used to identify purified pVIII-CBD-Ek-Oxa1p-8His fusion protein. All samples were prepared for electrophoresis by addition of SDS sample buffer (New England Biolabs, Inc. (NEB) #B7703S, Ipswich Mass.) followed by heating at 37° C. for 5 min.

Lane (M) contains a biotinylated protein ladder (Cell Signaling Technology #7727S, Beverly, Mass.) detected by anti-biotin, HRP-linked antibody.

Lane (m) was loaded with a sample of the membrane preparation before complete solubilization with n-dodecyl-beta-D-maltopyranoside (DDM).

Lane (L) shows the composition of the load sample after DDM solubilization and removal of insoluble material by centrifugation.

Lane (ft) shows the composition of the Nickel-NTA resin supernatant (flow-through) after incubation with load sample.

Lane (e1) shows the eluted protein after addition of 80 μL elution buffer containing 400 mM imidazole.

Lane (e2) shows the composition of a second 80 μL elution fraction. The arrow indicates the purified pVIII-CBD-Ek-Oxa1p-8His protein with calculated molecular weight of 62247 daltons.

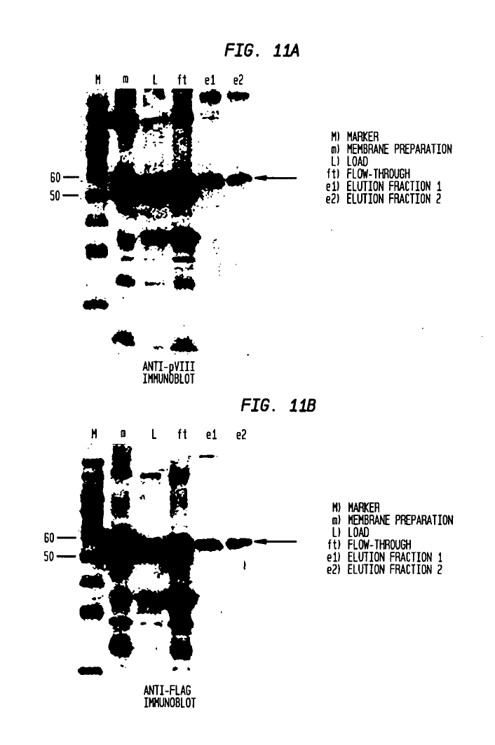

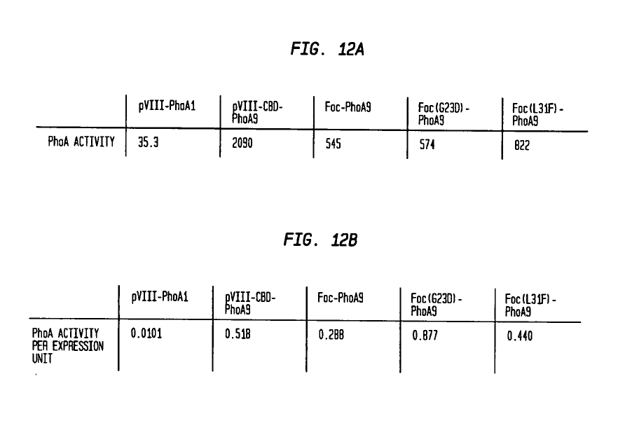

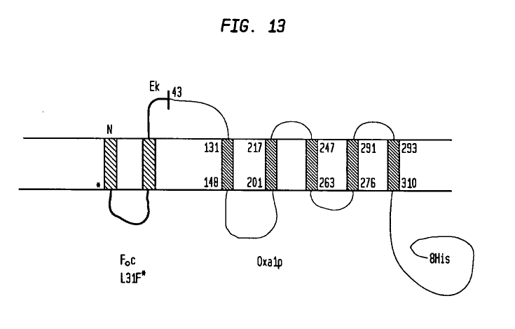

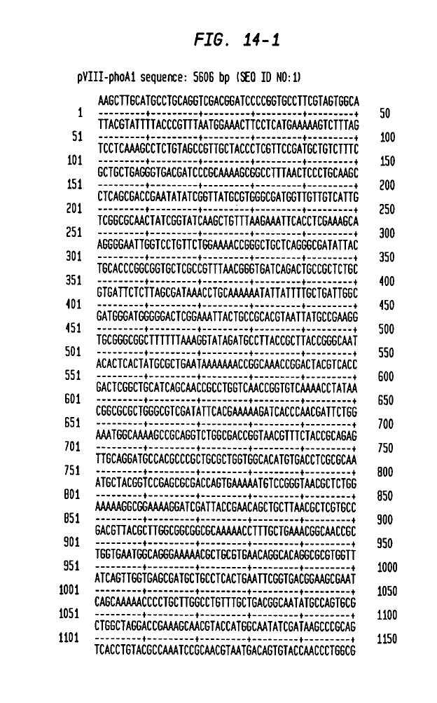

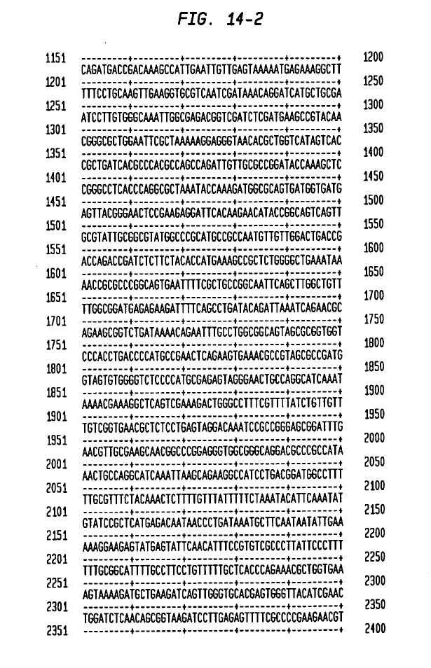

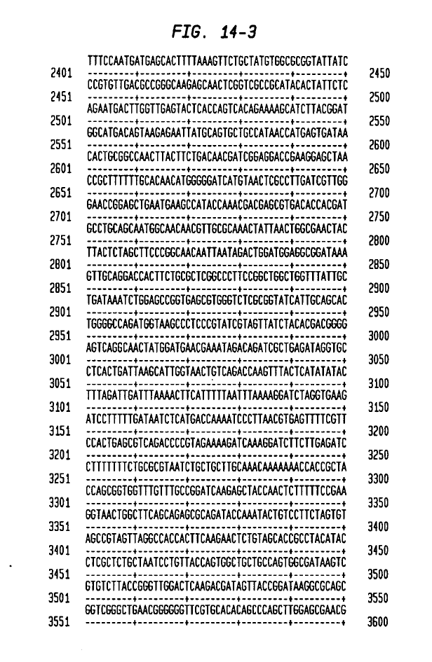

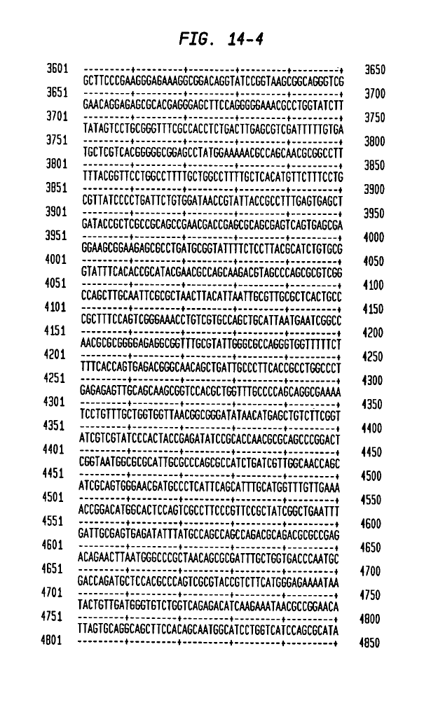

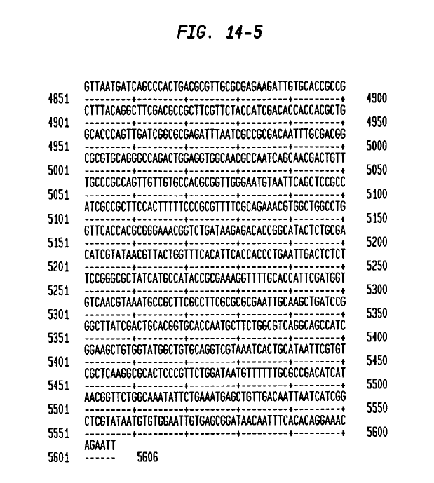

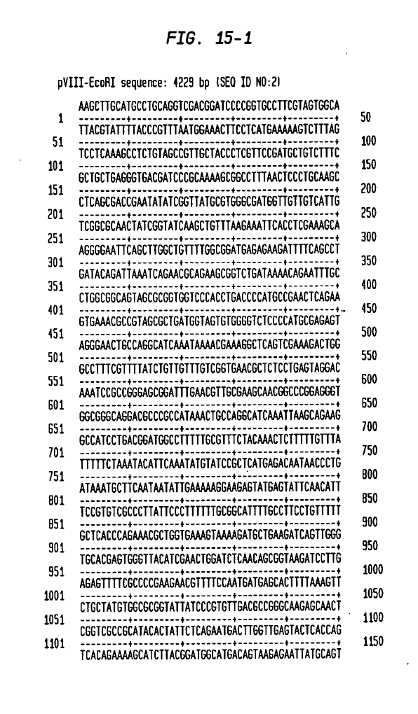

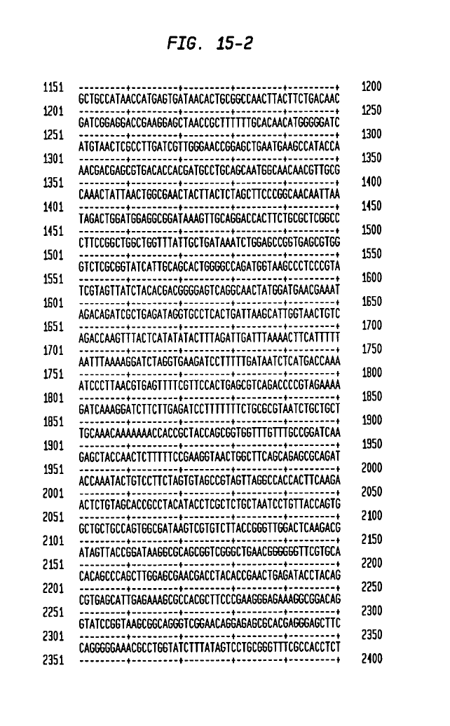

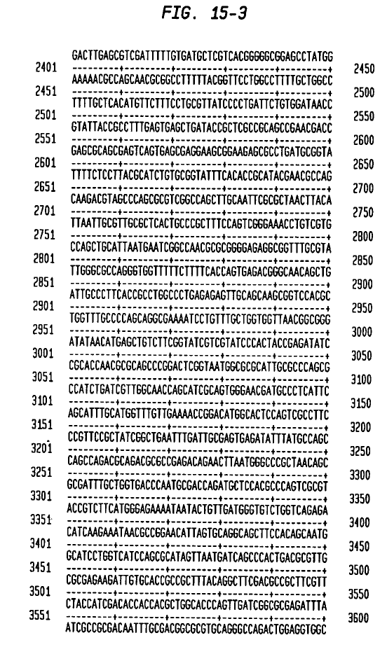

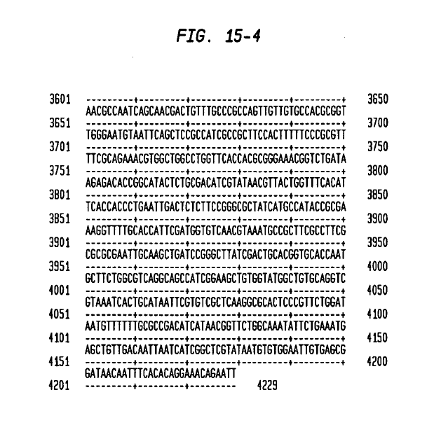

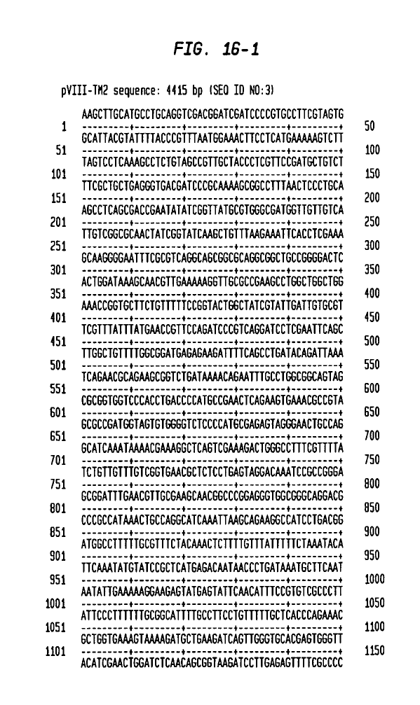

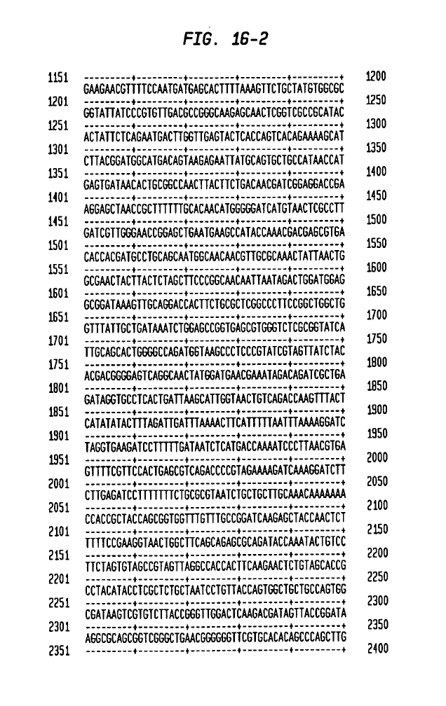

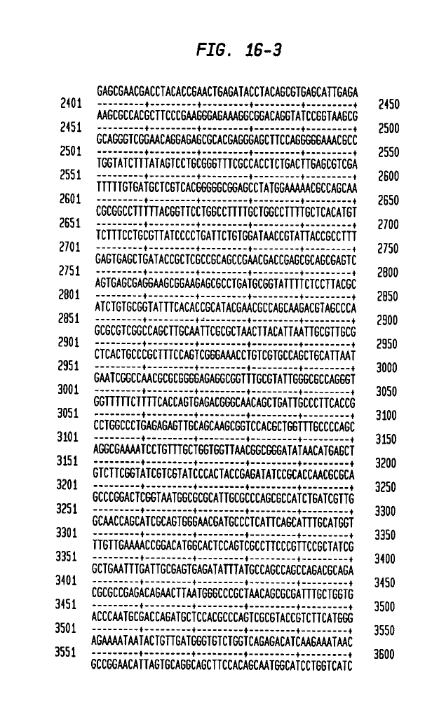

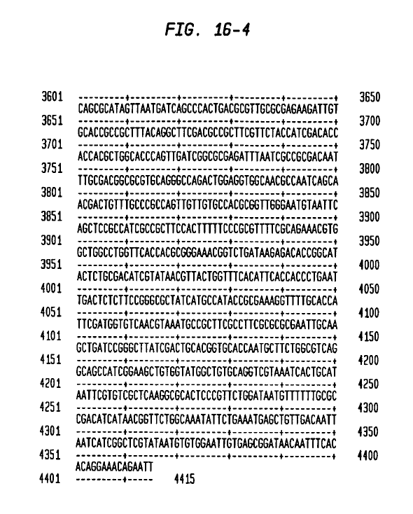

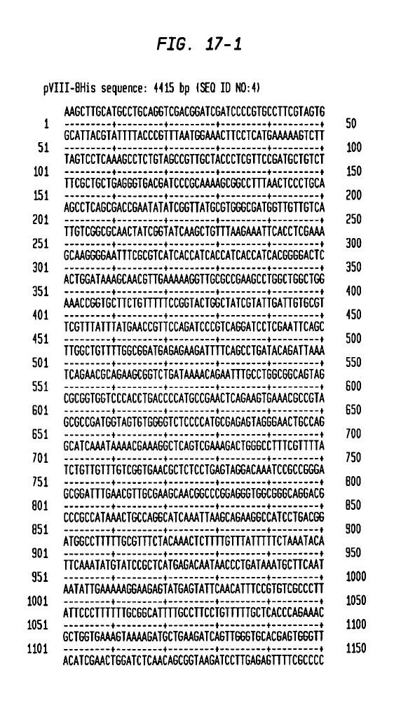

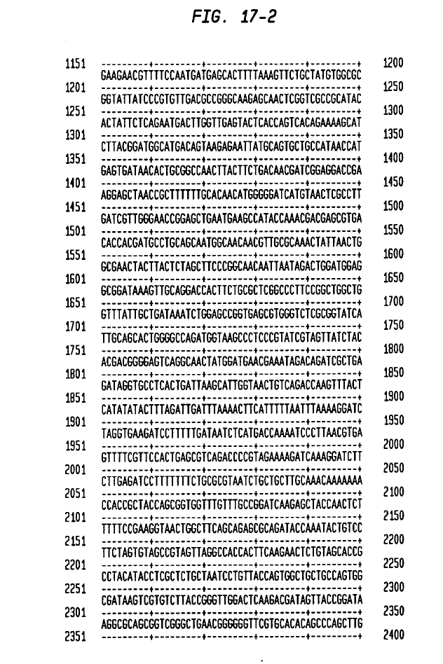

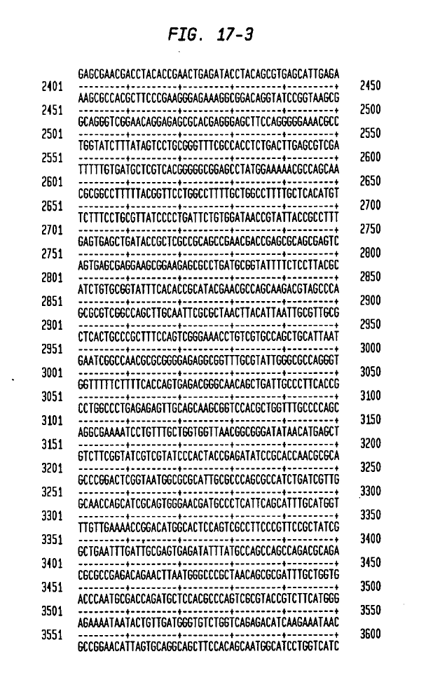

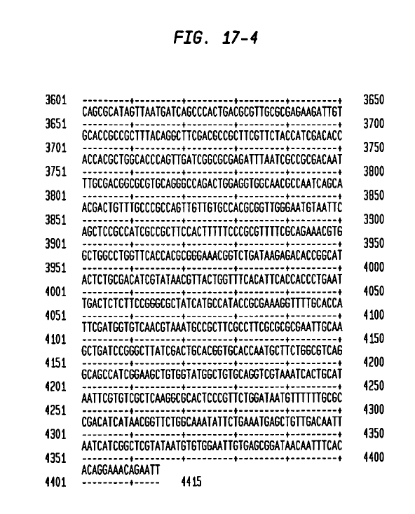

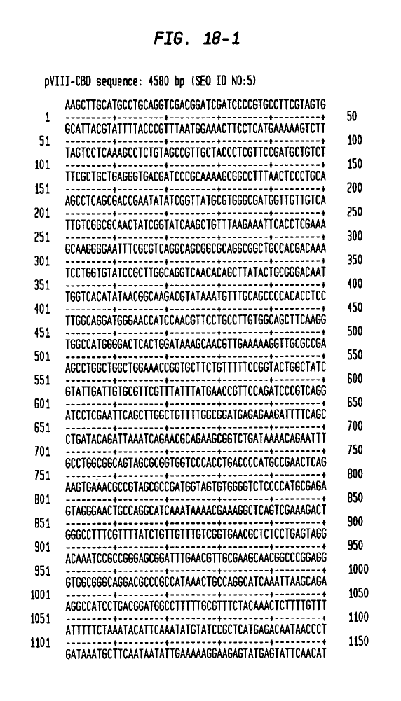

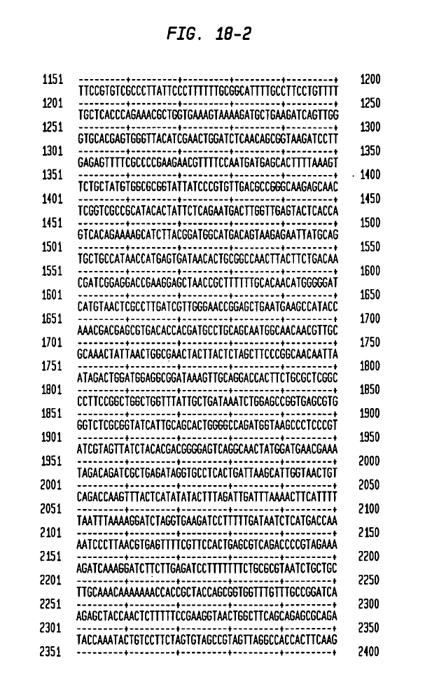

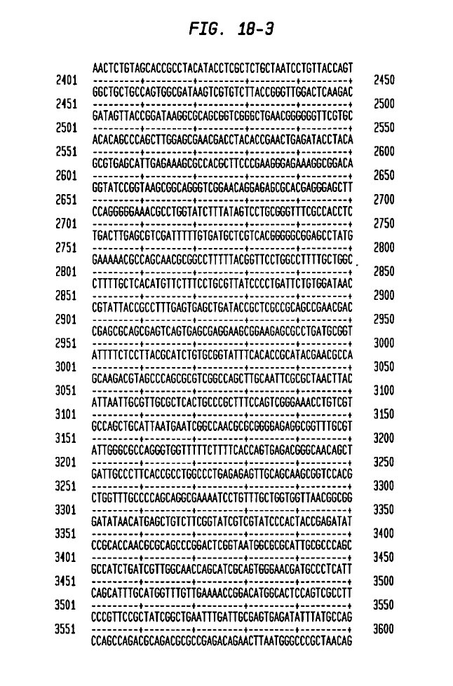

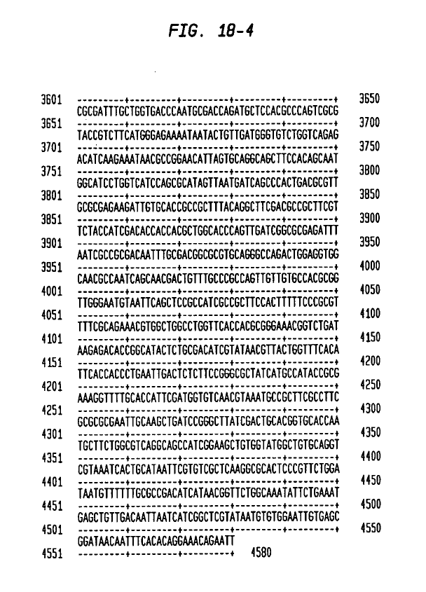

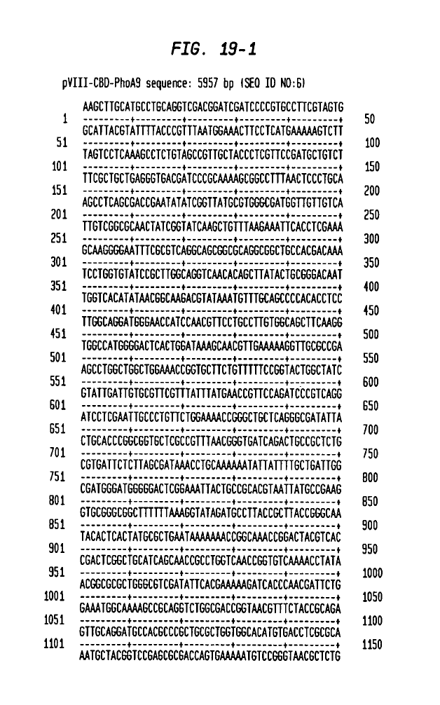

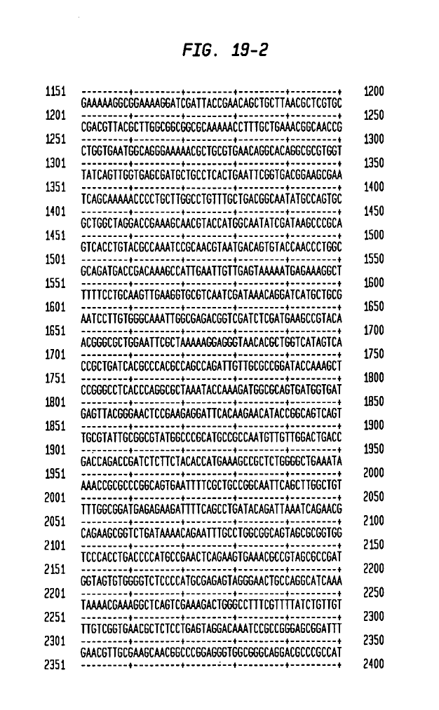

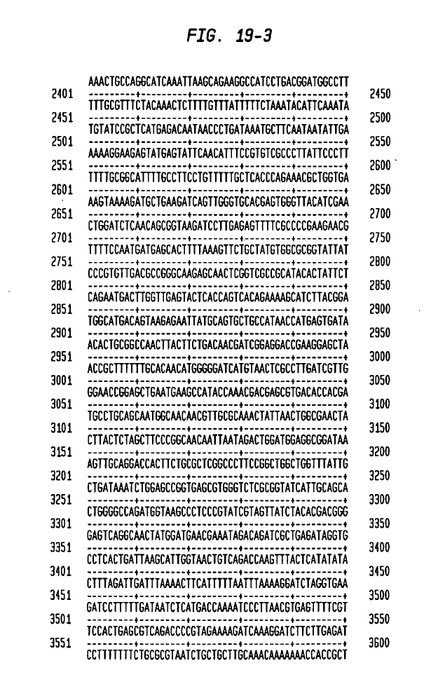

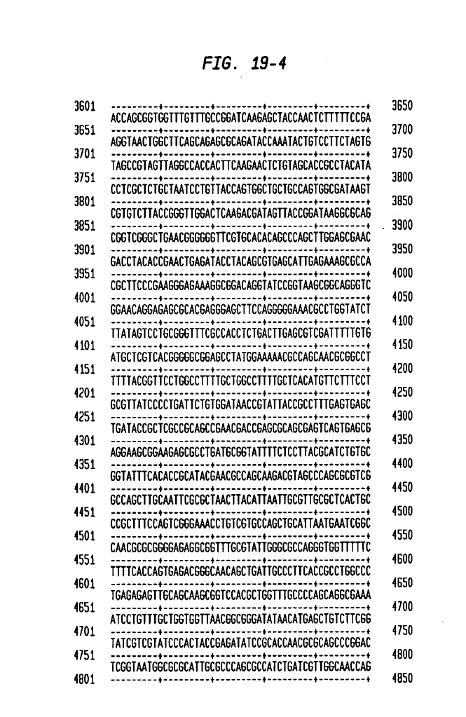

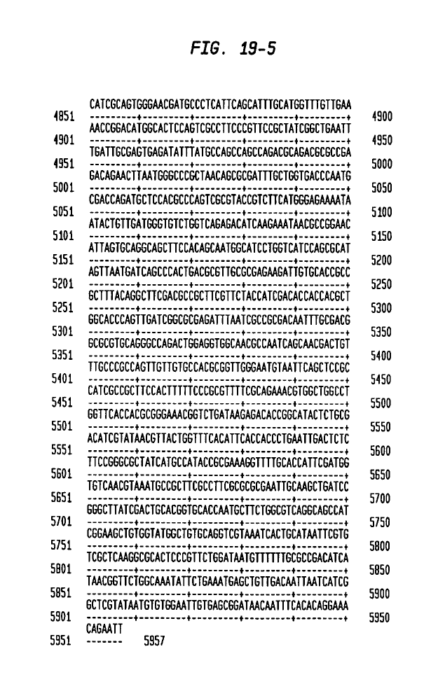

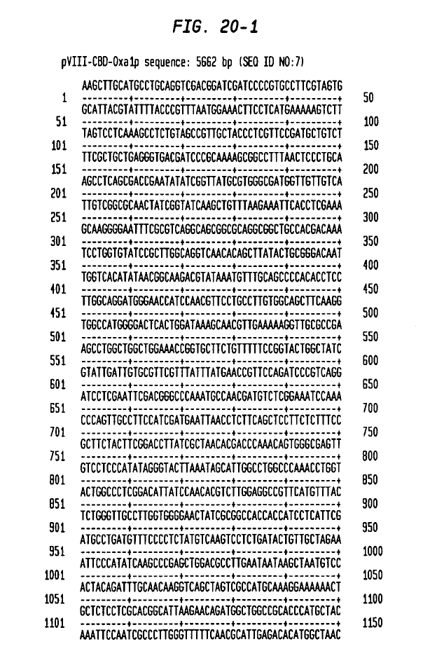

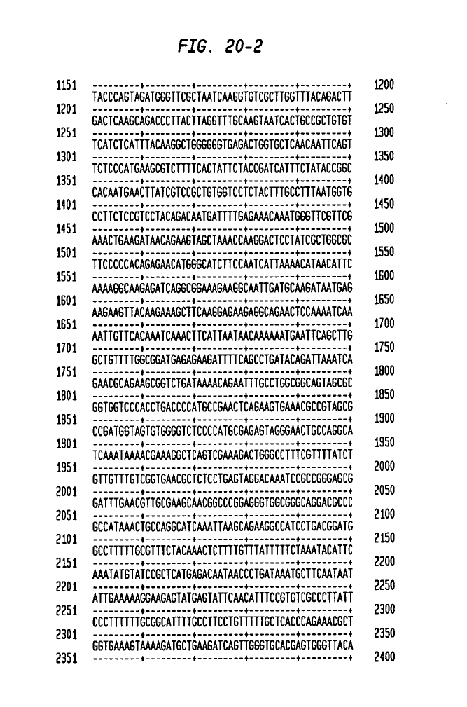

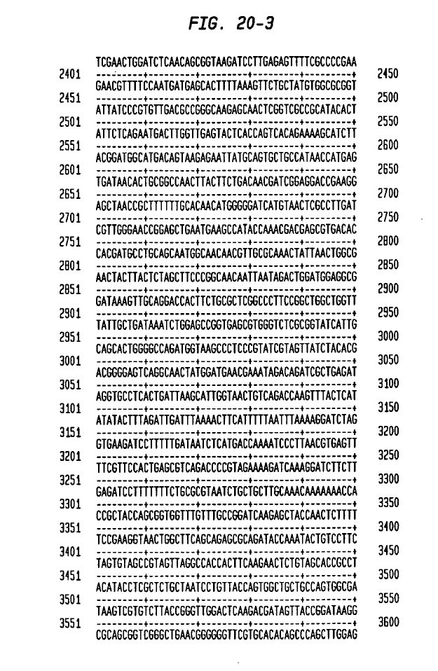

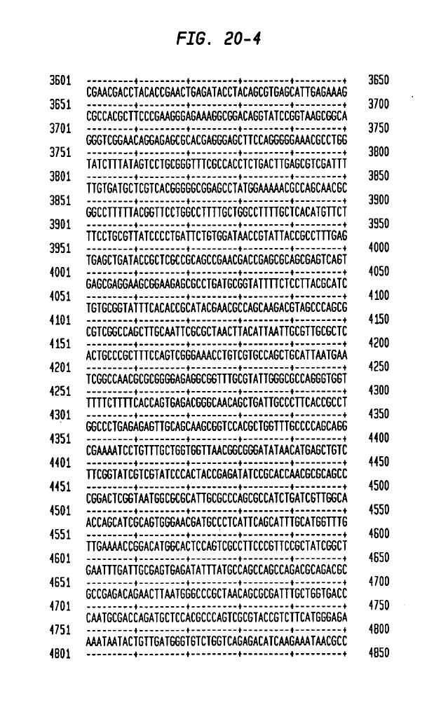

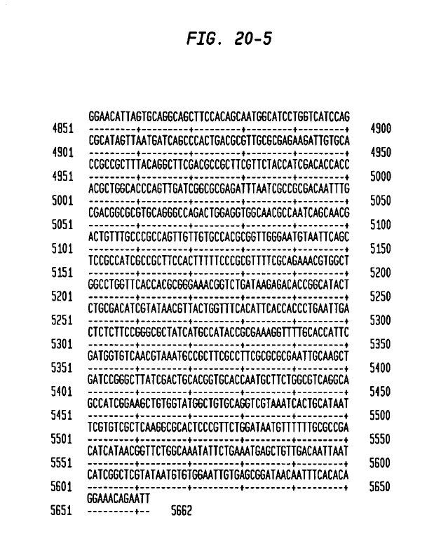

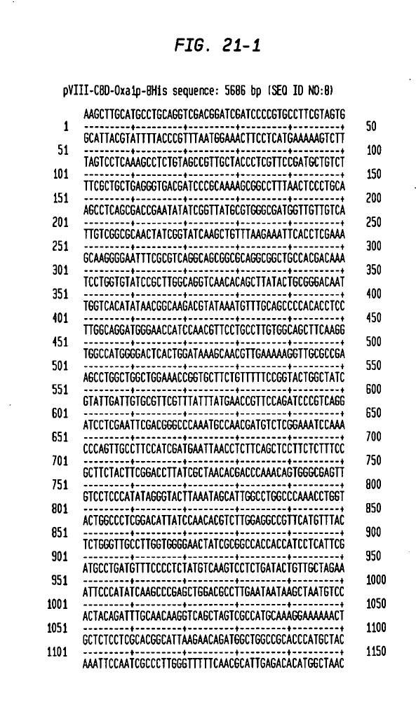

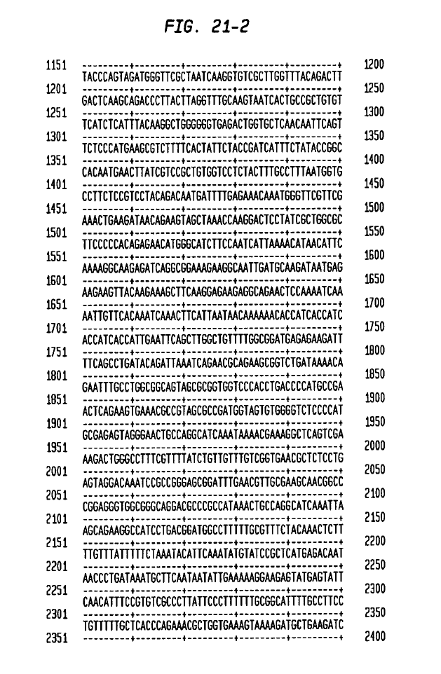

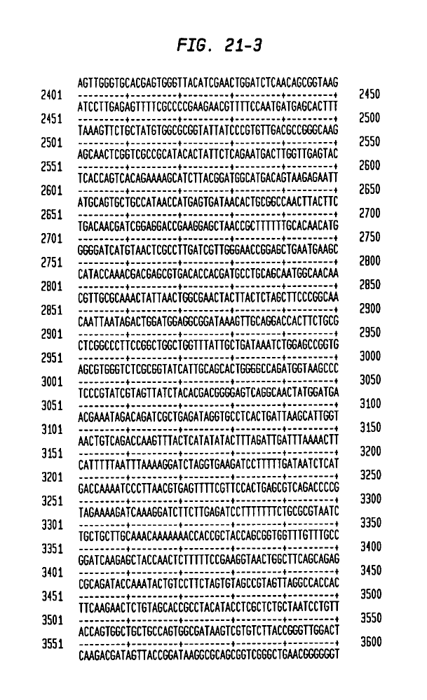

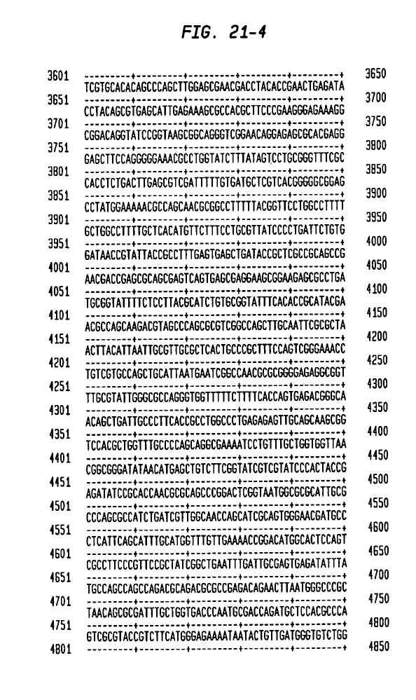

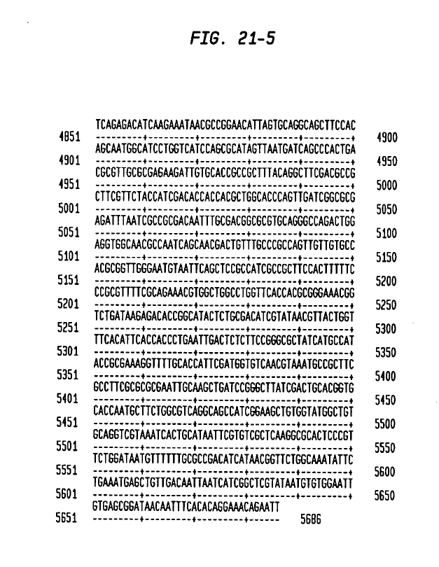

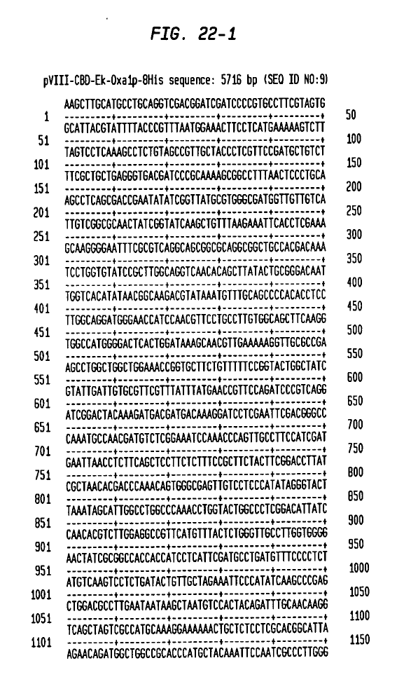

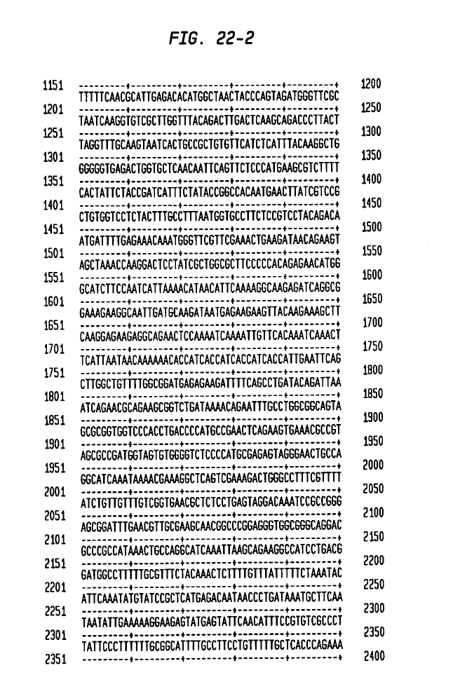

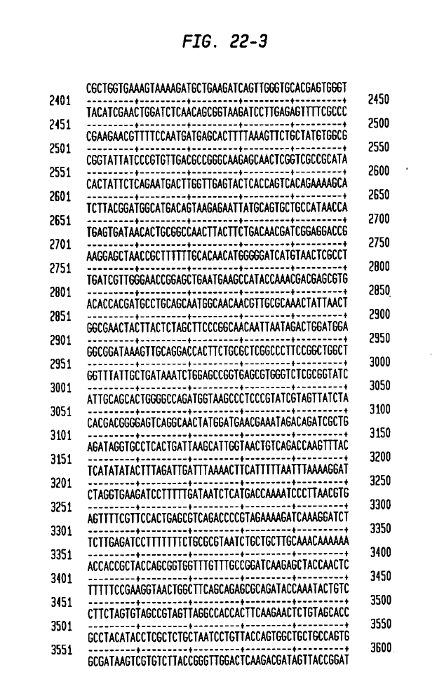

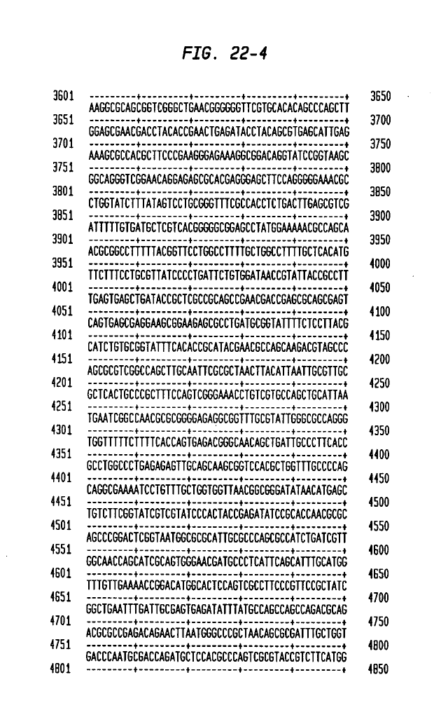

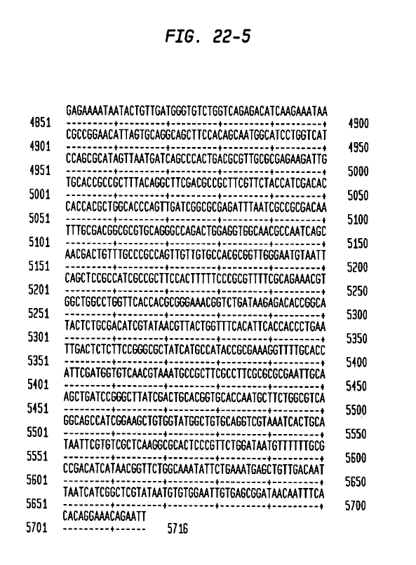

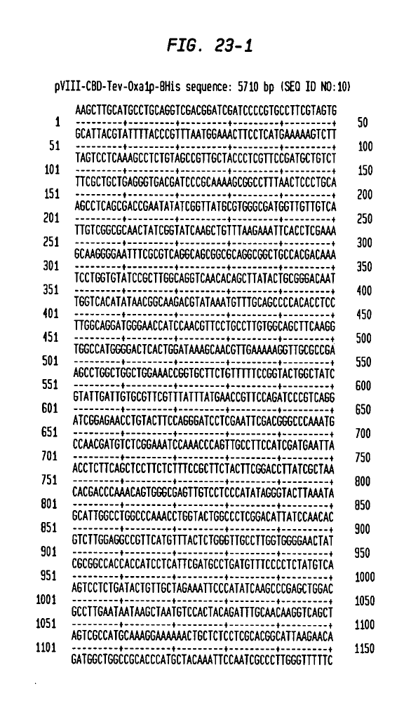

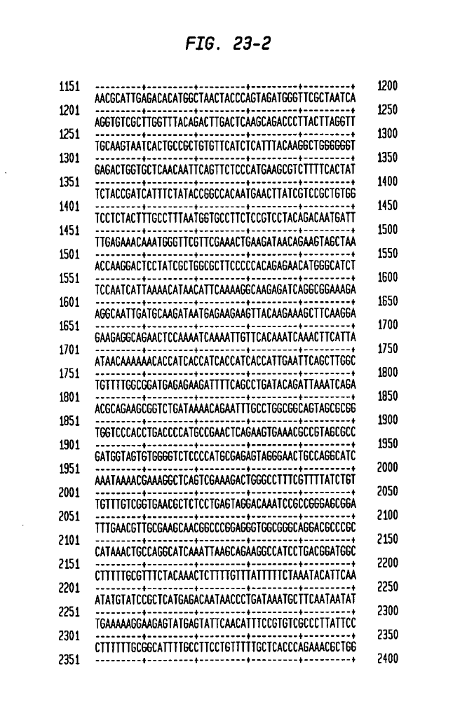

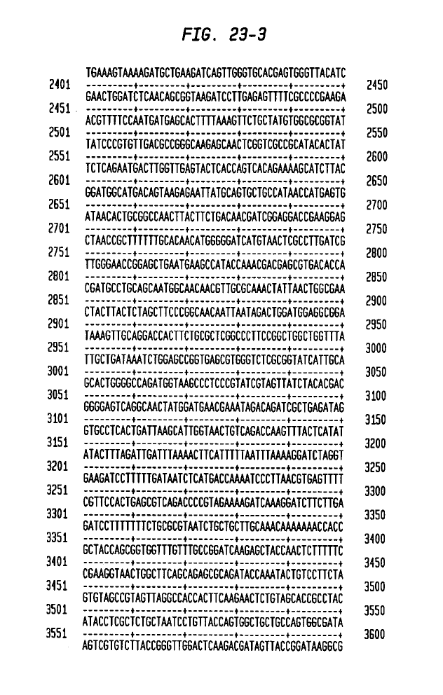

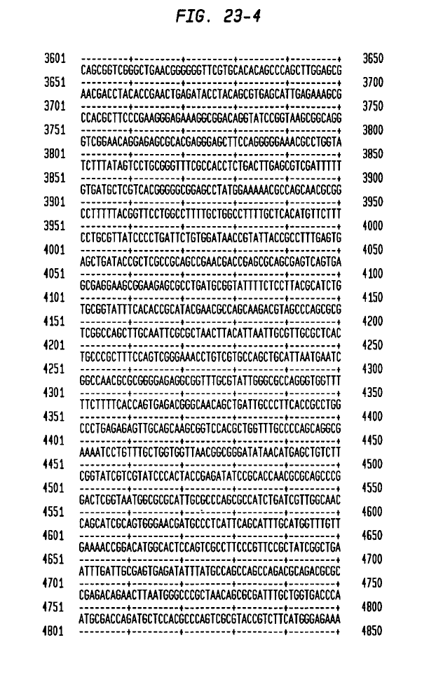

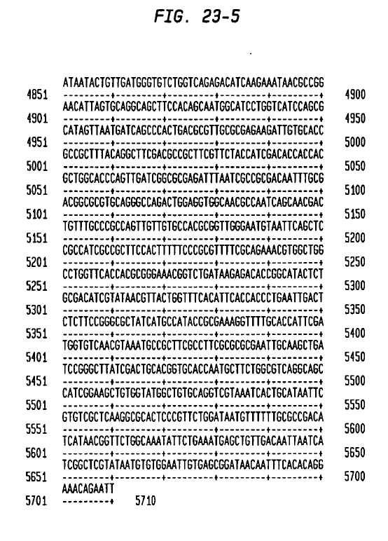

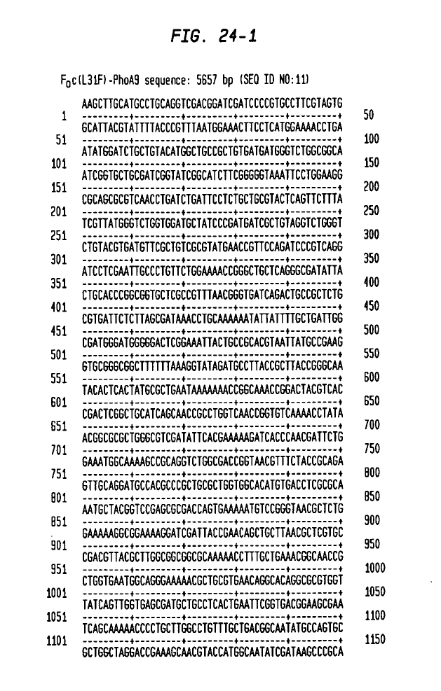

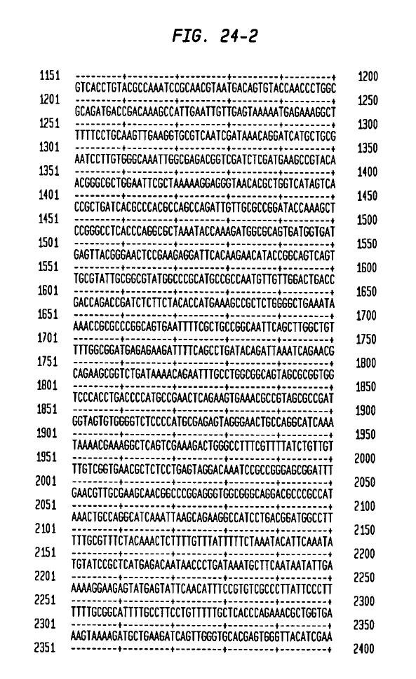

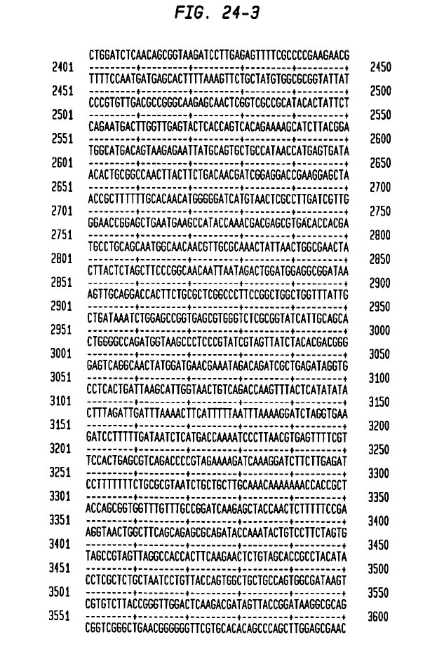

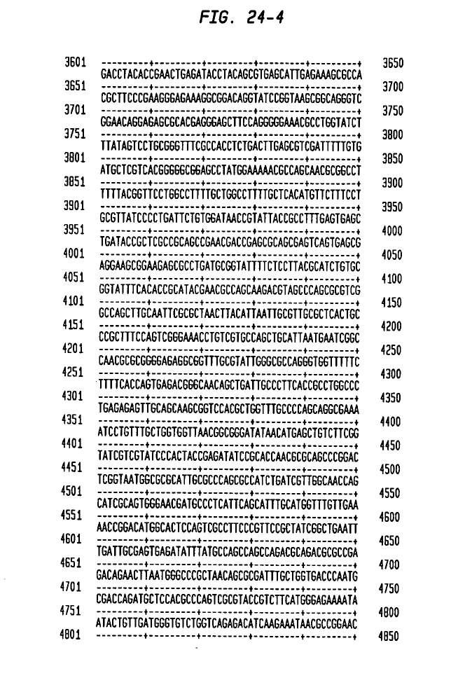

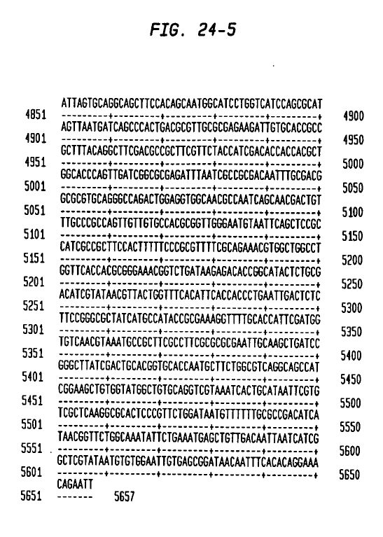

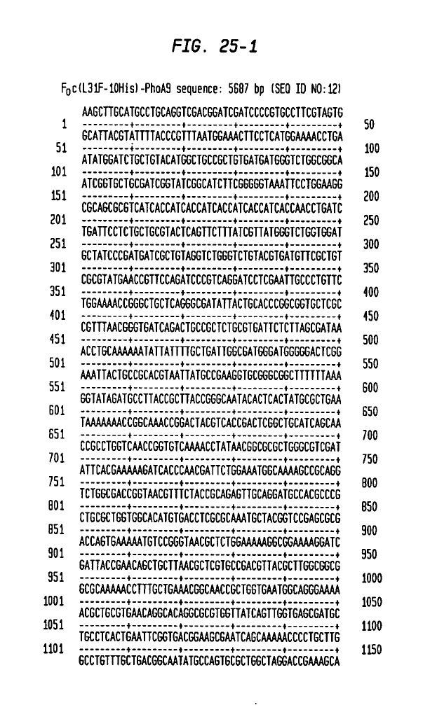

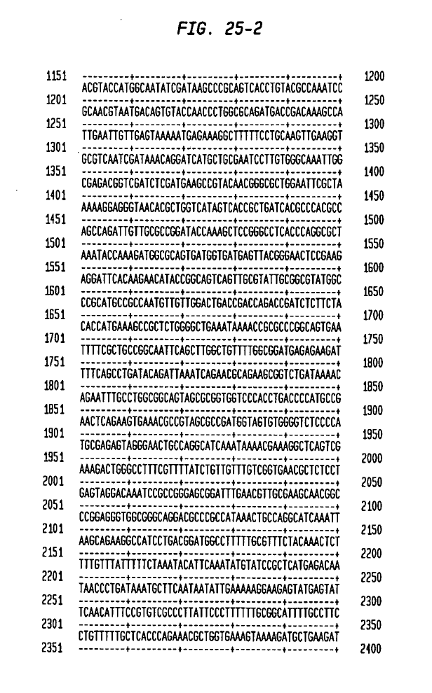

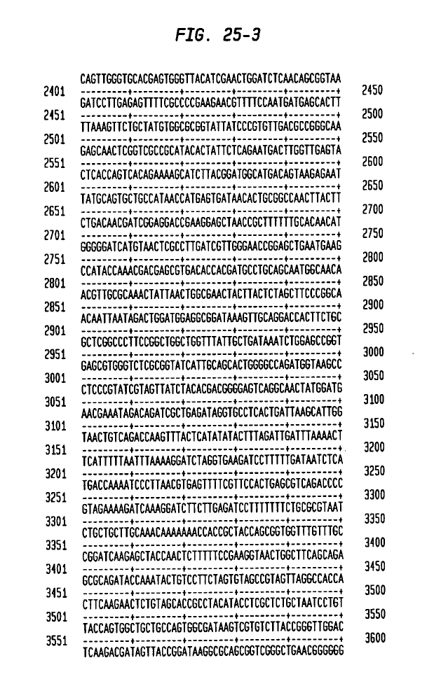

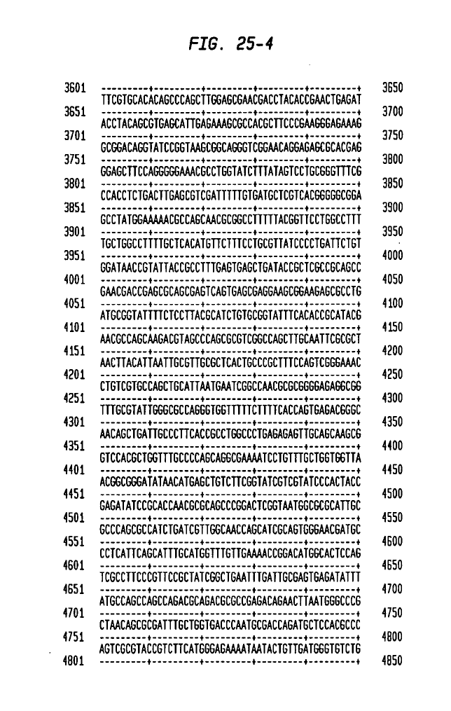

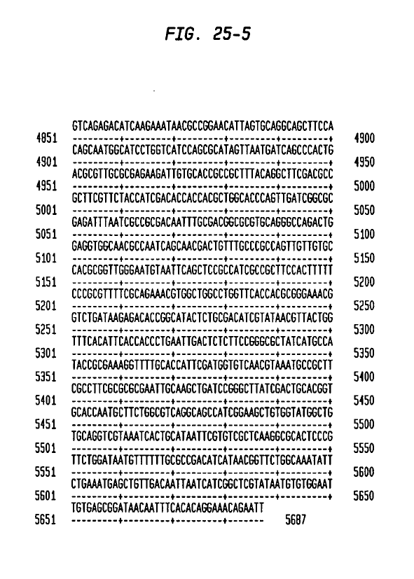

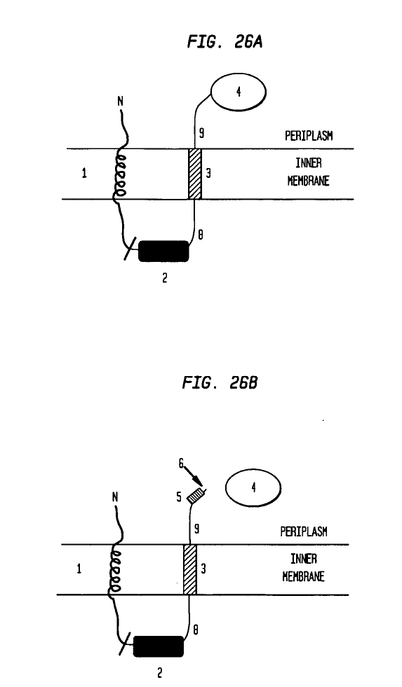

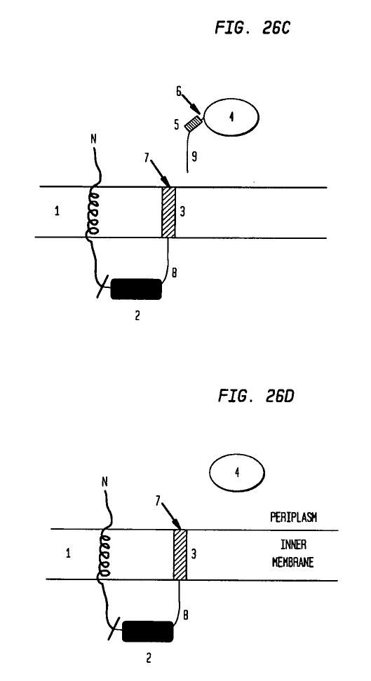

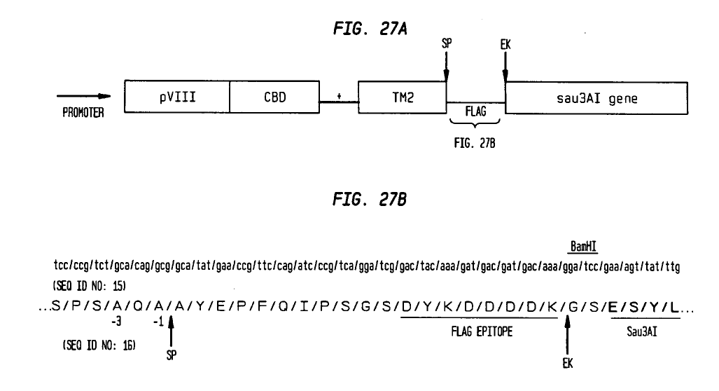

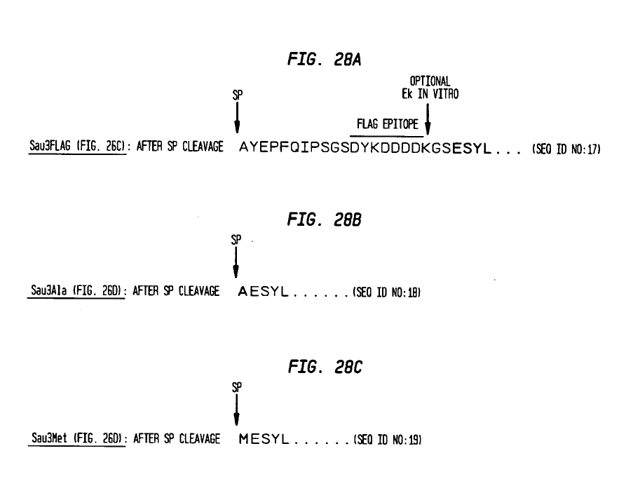

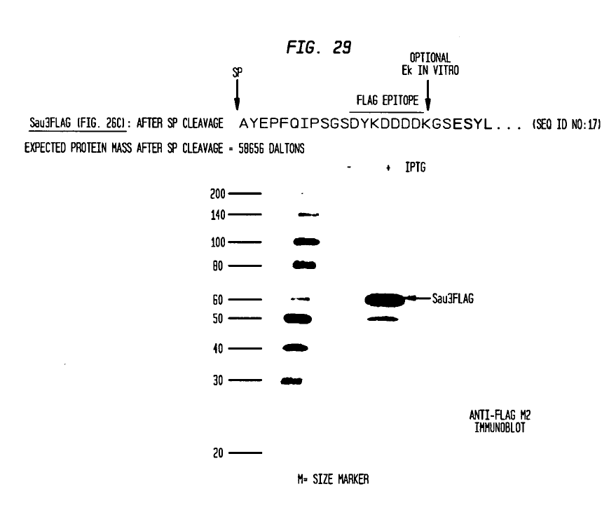

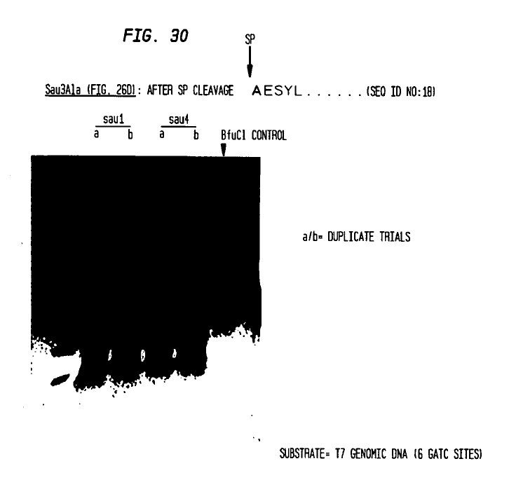

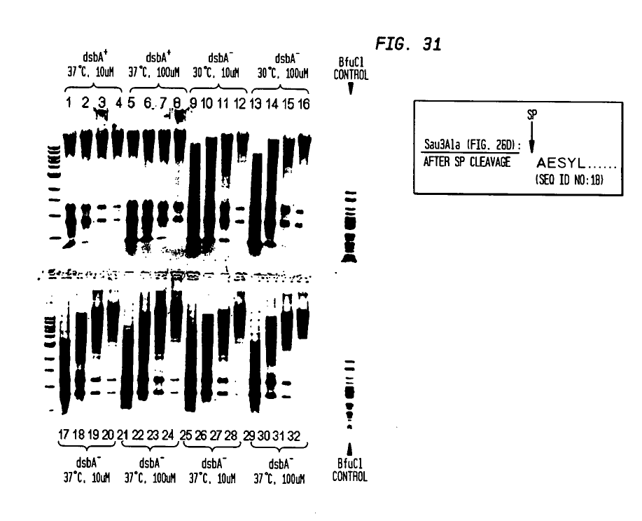

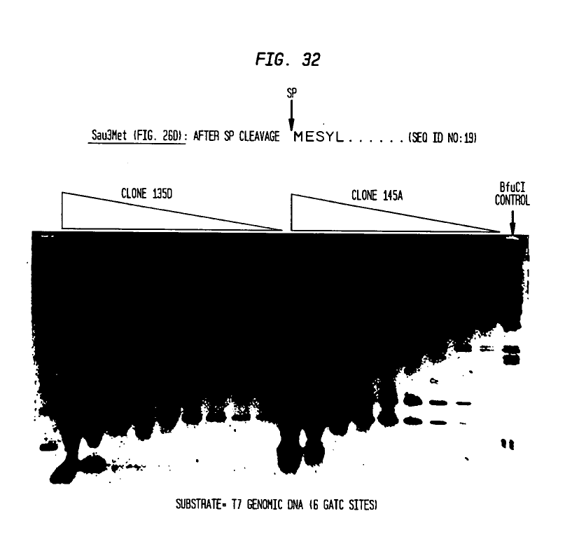

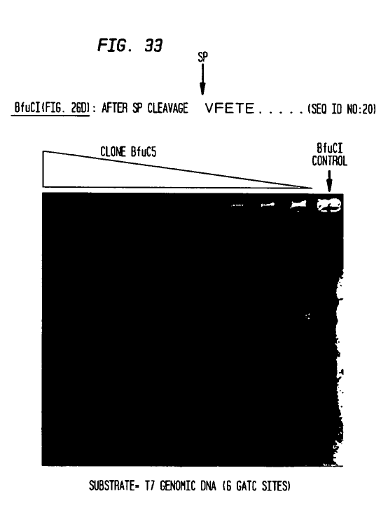

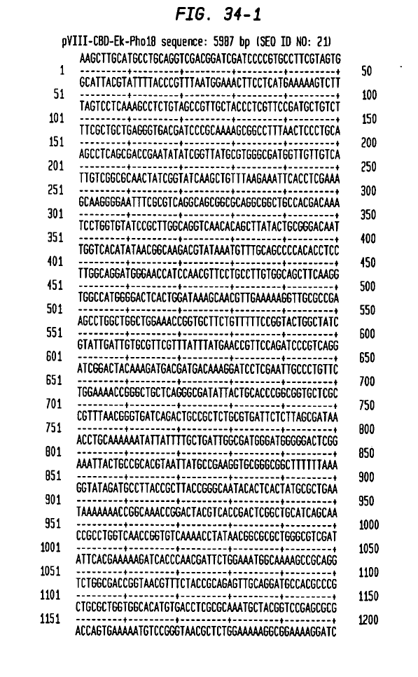

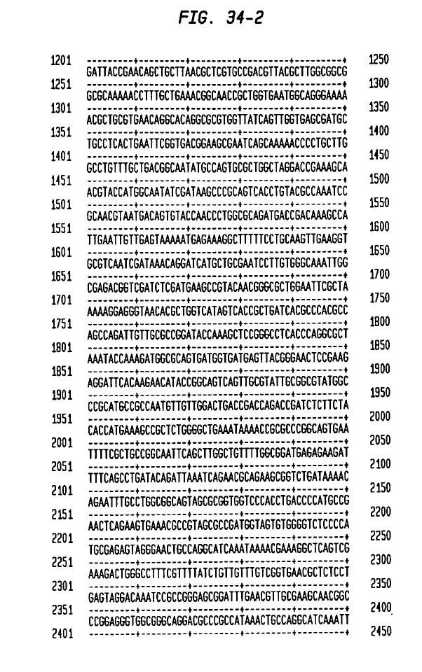

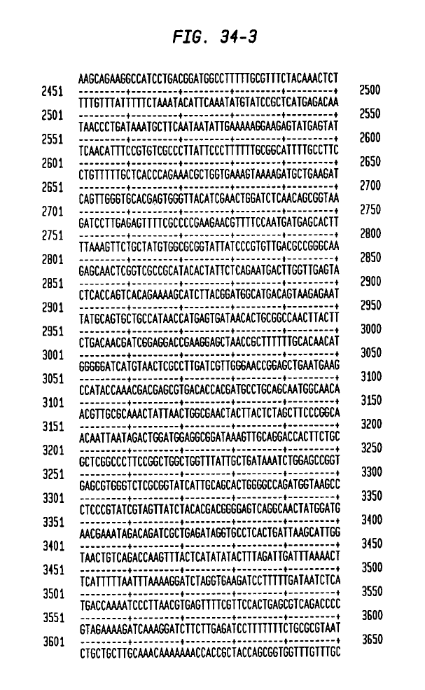

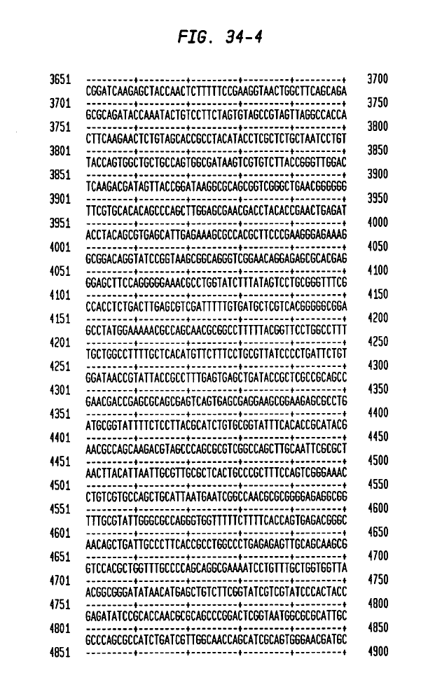

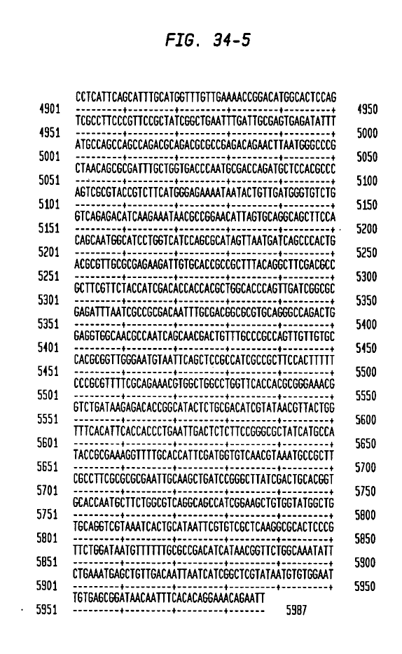

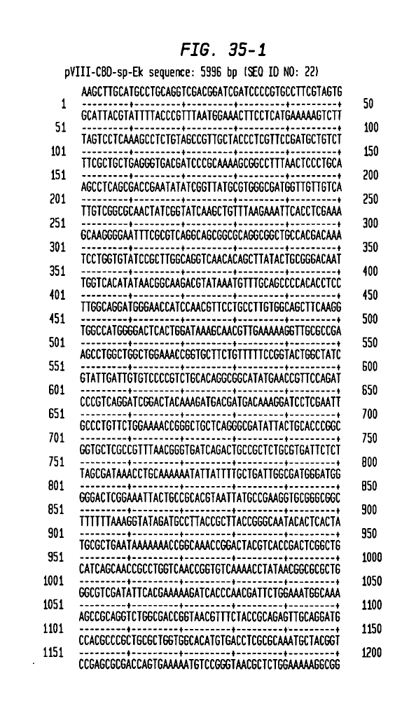

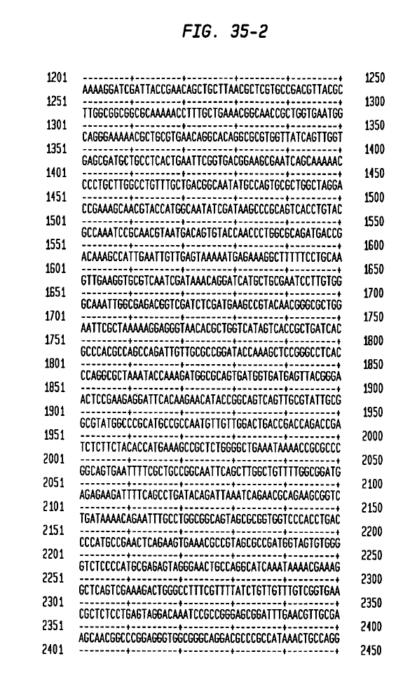

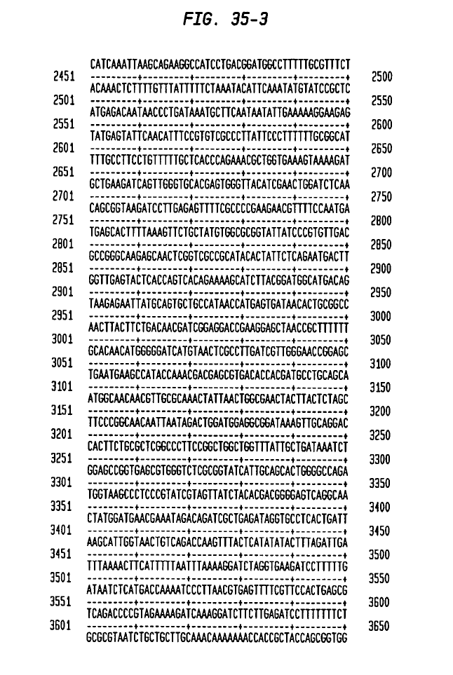

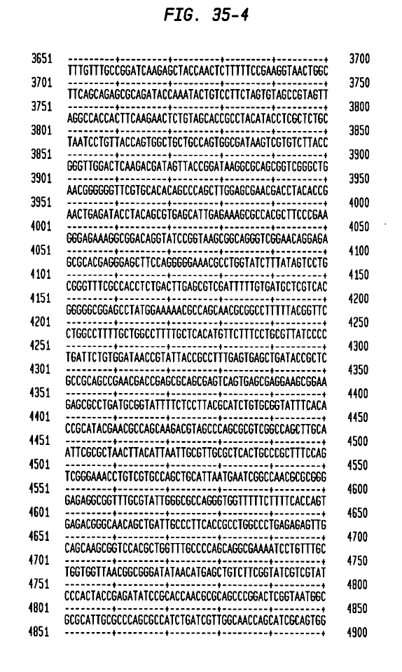

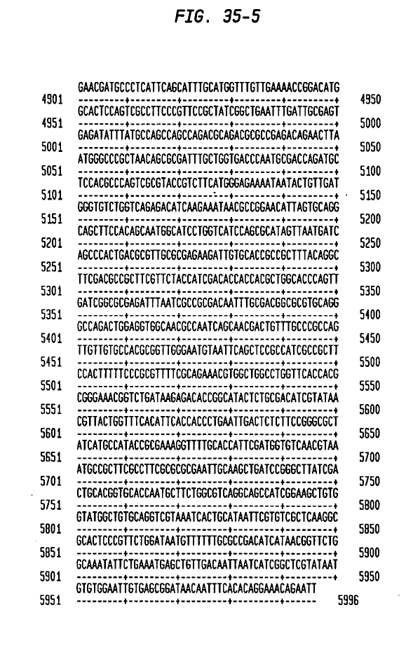

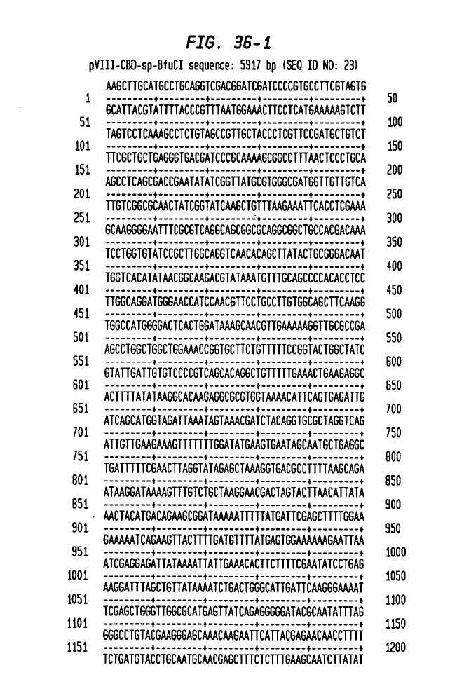

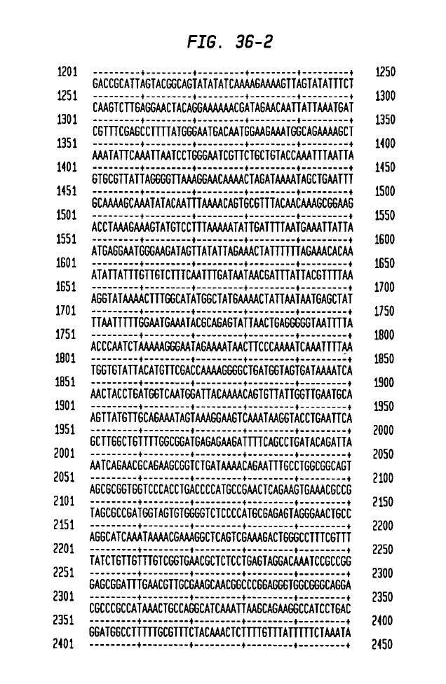

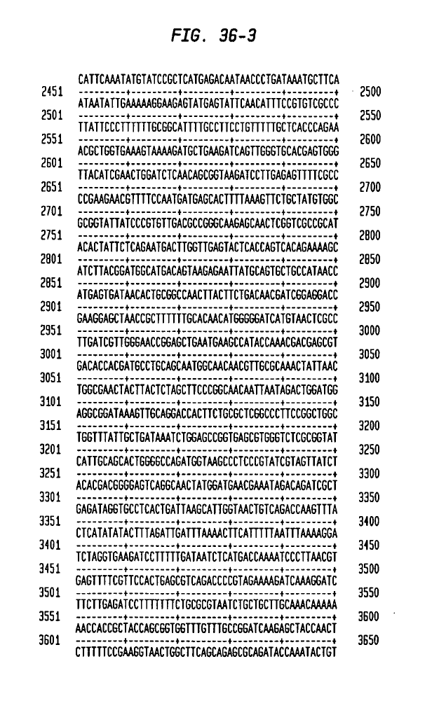

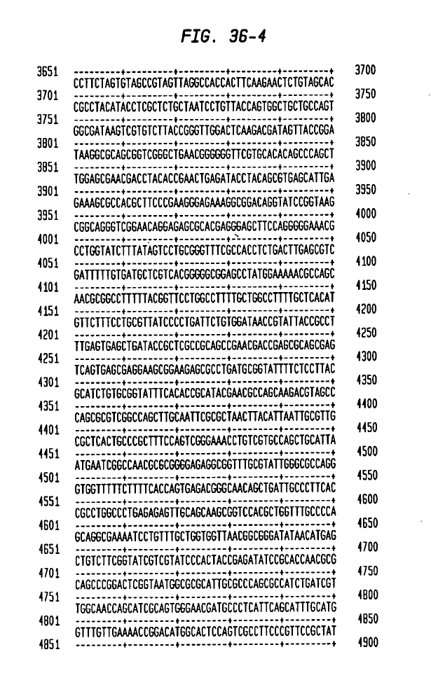

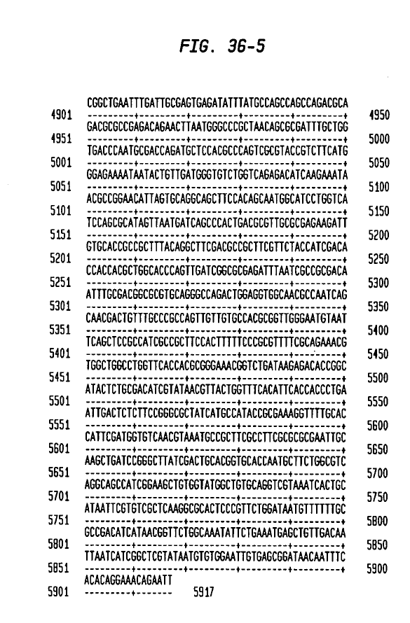

FIG. 11B shows an immunoblot where anti-FLAG monoclonal antibody was used to confirm purification of pVIII-CBD-Ek-Oxa1p-8His fusion protein. The lane descriptions from FIG. 11A apply to FIG. 11B. The arrow indicates the purified pVIII-CBD-Ek-Oxa1p-8His protein with calculated molecular weight of 62247 daltons.

FIGS. 12A and 12B are tables showing PhoA activity for different constructs (FIG. 12A) and PhoA activity per expression unit (FIG. 12B). DHB4 cells were grown at 30° C. and induced for 1 hr with 1 mM IPTG to express the exemplified fusion proteins.

FIG. 13 shows the membrane topology of a mutant subunit C F₀c(L31F) fused to a eukaryotic membrane protein (Oxa1p).

FIG. 14 shows the pVIII-PhoA1 sequence: 5606 bp (SEQ ID NO:1).

FIG. 15 shows the pVIII-EcoRI sequence: 4229 bp (SEQ ID NO:2).

FIG. 16 shows the pVIII-TM2 sequence: 4415 bp (SEQ ID NO:3).

FIG. 17 shows the pVIII-8His sequence: 4415 bp SEQ ID NO:4).

FIG. 18 shows the pVIII-CBD sequence: 4580 bp (SEQ ID NO:5).

FIG. 19 shows the pVIII-CBD-PhoA9 sequence: 5957 bp (SEQ ID NO:6).

FIG. 20 shows the pVIII-CBD-Oxa1p sequence: 5662 bp (SEQ ID NO:7).

FIG. 21 shows the pVIII-CBD-Oxa1p-8His sequence: 5686 bp (SEQ ID NO:8).

FIG. 22 shows the pVIII-CBD-Ek-Oxa1p-8His sequence: 5716 bp (SEQ ID NO:9).

FIG. 23 shows the pVIII-CBD-Tev-Oxa1p-8His sequence: 5710 bp (SEQ ID NO:10).

FIG. 24 shows the F₀c(L31F)-PhoA9 sequence: 5657 bp (SEQ ID NO:11).

FIG. 25 shows the F₀c(L31F-10His)-PhoA9 sequence: 5687 bp (SEQ ID NO:12).

FIG. 26 shows cartoons of different types of N-terminal protein vehicles for envelope-targeting. (1) corresponds to a membrane-targeting peptide that extends through the membrane into the cytoplasm and optionally into the extracellular space. (2) corresponds to a cytoplasmic protein domain, where the protein domain is capable of binding to an affinity substrate. (3) corresponds to a trans-membrane segment (TM2), the TM2 being linked to the affinity-binding domain via a protease-resistant cytoplasmic peptide (8). The protein of interest is (4). The extracellular epitope is (5). (9) is the linker sequence optionally containing a cleavage site for signal peptidase (SPase) (7) or a cleavage site for a heterologous protease (6).

FIG. 26A shows that (4) is tethered in the extra-cytoplasmic space.

FIG. 26B shows that (4) can ultimately be cleaved in vivo from (5) at a cleavage site (6) located between (5) and (4). This requires a strain expressing a heterologous protease for secretion into the periplasm in a regulated or constitutive manner. In vivo cleavage by a heterologous protease targeted to the periplasm may involve for example, Enterokinase (Ek) or Tobacco Etch Virus (Tev).

FIG. 26C shows that (4) attached to (5) can ultimately be cleaved at an SPase recognition sequence (7) between (3) and (5), leaving the extracellular epitope linked to the protein of interest (4). The host cell inherently contains SPase hence in vivo cleavage is constitutive.

FIG. 26D shows a protein fusion in which the SPase cleaves directly between (3) and (4) at (7) in the absence of (5).

FIGS. 27A and 27B show a gene fusion combining desirable elements for protein trans-membrane export in which the pVIII-CBD vector may be employed for export of a protein of interest and released into the periplasm by constitutive cleavage by E. coli SPase.

FIG. 27A shows a vector map of a pVIII construct. This includes a promoter of choice (Ptac), a DNA coding for a membrane-targeting peptide (pVIII), a DNA coding for an affinity-binding protein (CBD), a DNA coding for a TM2, an optional SPase site, an optional DNA encoding an epitope (FLAG), an optional DNA encoding a protease cleavage site (Ek) and a DNA encoding a protein of interest.

FIG. 27B shows a linear diagram of a cloning strategy where the exported protein may be expressed with an N-terminal FLAG epitope after in vivo cleavage by E. coli SPase. This modification of the pVIII-CBD vector is designated as pVIII-CBDspEk. The abbreviation “sp” indicates an SPase site. The abbreviation “Ek” indicates the inclusion of the FLAG epitope (DYKDDDDK) (SEQ ID NO:13) and corresponding Enterokinase protease cleavage site (DDDDK) (SEQ ID NO:14).

FIGS. 28A-C show cloning and export strategies for Sau3AI in E. coli.

FIG. 28A: Sau3FLAG after SPase cleavage having a configuration shown in FIG. 26C (SEQ ID NO:17).

FIG. 28B: Sau3Ala after SPase cleavage having a configuration as shown in FIG. 26D (SEQ ID NO:18).

FIG. 28C: Sau3Met after SPase cleavage having a configuration as shown in FIG. 26D (SEQ ID NO:19).

FIG. 29 shows Sau3AI export. The expected protein mass after SPase cleavage was 58656 daltons. After SPase cleavage, Sau3AI protein fused to FLAG was obtained as detected by immunoblot of the FLAG epitope using anti-FLAG M2 where the N-terminal amino acids are AYEPFQIPSGSDYKDDDDKGSESYL . . . , (SEQ ID NO:17).

FIG. 30 shows expression of Sau3AI in a non-methylase protected E. coli dsbA⁻ host MB68. The DNA substrate is T7 genomic DNA (6 GATC sites). After SPase cleavage, the N-terminal end of the Sau3AI protein was AESYL . . . , (SEQ ID NO:18). The results of duplicate trials are shown in lanes a and b as labeled. The left-hand lane is a size marker while the right-hand lane is the product of digestion by BfuCI which serves as a control.

FIG. 31 shows expression of Sau3AI in E. coli dsbA⁻ mutant. After SPase cleavage, the protein has an N-terminal end: AESYL . . . , (SEQ ID NO:18). The lanes are labeled and are replicated four times for each sample. From left to right, the results of substrate cleavage are shown at 37° C. using 6 units purified enzyme and a BfuCI enzyme serves as a control.

The dsbA⁻ heading indicates protein expression was carried out in strain MB68 while the heading dsbA⁺ indicates protein expression was carried out in the wild type (wt) dsbA strain DHB4 (isogenic parent strain of MB68). Each lane shows the results of digesting 1 μg phage lambda genomic DNA with either 2 μl of cell lysate or 2 μl of diluted cell lysate. Thus each set of 4 lanes represents a 2-fold serial dilution series. The temperature within the heading indicates the culture outgrowth temperature while the concentration indicates the final IPTG concentration used to induce Sau3AI expression. All cultures were shifted to 20° C. during the Sau3AI induction period. The right-hand lane is the product of digestion by BfuCI which serves as a control.

FIG. 32 shows the export of wt Sau3AI using pVIII-CBDsp in the dsbA⁻ E. coli strain MB68. Sau3AI was expressed from either clone 135D or 145A. The substrate is 1 μg of T7 genomic DNA (6 GATC sites). After SPase cleavage, the Sau3AI has an N-terminal sequence of MESYL . . . , (SEQ ID NO:19). The results with 2 different clones are shown. The left-most lane is a 1 kb marker (NEB, Ipswich, Mass.). The right-most lane is the BfuCI digest control. A two-fold serial dilution is shown of the lysate of each clone. Each series of lanes represent reactions resulting from a 2-fold series dilution of cell lysate (beginning with 2 μl of undiluted lysate).

FIG. 33 shows export of BfuCI using pVIII-CBDsp in the dsbA⁻ E. coli strain MB68. The substrate is T7 genomic DNA (6 GATC sites). After SPase cleavage, the N-terminal end of the BfuCI is VFETE . . . , (SEQ ID NO:20). The right-most lane is a BfuCI control digest.

FIG. 34 shows the pVIII-CBD-Ek-Pho18 DNA sequence (SEQ ID NO:21).

FIG. 35 shows the pVIII-CBD-sp-Ek DNA sequence (SEQ ID NO:22).

FIG. 36 shows the pVIII-CBD-sp-BfuCI DNA sequence (SEQ ID NO:23).

### DETAILED DESCRIPTION OF THE EMBODIMENTS

Present embodiments of the invention provide methods and compositions for expression and transport of heterologous membrane proteins (proteins of interest) to the inner membrane, and membrane translocation of heterologous soluble proteins including toxic proteins to the periplasm of host cells. The methods include expression of DNA encoding N-terminal protein vehicles fused to DNA encoding proteins of interest. The expressed fusion proteins are expressed in the cytoplasm and targeted to the inner membrane in a YidC-dependent and/or SRP-dependent manner.

The compositions include recombinant DNA which encodes an N-terminal protein vehicle and the protein of interest. The N-terminal protein vehicle preferably includes a membrane-targeting peptide, a cytoplasmic affinity-binding domain, and a trans-membrane segment. In addition, the N-terminal protein vehicle may contain an SPase site such that the in vivo cleavage by an SPase releases the soluble protein of interest or releases the fused membrane protein of interest. Additionally or alternatively, the N-terminal protein vehicle may include a heterologous protease site such as a Tev protease cleavage site or an Enterokinase cleavage site for in vivo or in vitro cleavage and release of the protein of interest.

The expression of the genetically engineered DNA occurs in a host cell that is suited for expression of the protein of interest. For example, toxic soluble proteins that contain cysteines and do not require disulfide bonds for folding and activity are preferably expressed in dsbA⁻ host cells. Using embodiments of the method described above, toxic restriction enzymes were successfully expressed and exported to the periplasm in active form (without modification of host genomic DNA with a protective group such as cytosine or adenine methylation) without toxic effects to the host cell.

### The Protein of Interest

The “protein of interest” refers to a desired recombinant protein for integration within the host bacterial cell inner membrane or for translocation across the inner membrane into the periplasmic space.

Examples of proteins of interest include: DNA metabolizing enzymes that negatively affect host cell viability (e.g. restriction enzymes) and other cytotoxic proteins; proteases that affect host cell physiology (e.g., ATP-dependent proteases); proteins that are normally secreted in native host, but where expression and export are unknown in E. coli (e.g. alpha-1,3-galactosidase from Xanthomonas manihotis); and proteins that are prone to aggregation/inclusion body formation when expressed in the cytoplasm (e.g., peripheral membrane proteins and proteins normally residing in multi-protein complexes). Such proteins often possess a hydrophobic protein-interaction surface, which causes aggregation when the protein is expressed alone without its partner. These proteins may be more stably expressed when targeted to the surface of the inner membrane. Anchoring such a protein to the inner membrane may aid in stable expression of the fused protein.

A protein of interest is here exemplified by P2, the polytopic integral membrane protein Oxa1p, or the secreted protein PhoA and also by toxic restriction endonucleases Sau3AI and BfuCI.

### Host Cells

Host cells for expressing fusion proteins are preferably gram negative bacteria such as E. coli which have an envelope. (An “envelope” refers to the bacterial cell structure that forms a barrier between the cytoplasmic space and the extracellular environment. This generally includes the inner membrane and periplasm of a bacterium.) The host cell preferably is a DsbA⁻ phenotype if the proteins have cysteines but do not require disulfide bonds for folding and activity. For example, Sau3AI contains 3 cysteines and is inactive when expressed into the periplasm in a wt dsbA strain but is active when expressed into the periplasm in a strain lacking periplasmic DsbA (DsbA⁻). Restriction enzyme BfuCI (2 cysteines) is active when expressed to the periplasm in a DsbA⁻ host cell.

While not wishing to be limited by theory, we propose that pVIIICBD-PhoA membrane-targeting peptide depends on signal recognition particles in E. coli host cells. This was demonstrated using cells containing wt signal recognition particle component Ffh (fifty-four homologue encoded by the ffh gene) or mutated Ffh (protein variant Ala37Pro expressed from the ffh77 allele) cells (mutation reported by Tian et al. (J Bacteriol. 184(1):111-8 (2002)) and Huber et al. (J Bacteriol. 187(9):2983-91 (2005)). We constructed an MC1061 derivative with the ffh77 mutation in order to examine the SRP-dependence of the pVIII-CBD N-terminal protein vehicle. MC1061 (ffh77) was derived from MC1061 using the allele exchange method described by Hamilton et al. (J Bacteriol. 171(9):4617-22 (1989)). When pVIII-CBD-Ek-PhoA18 was expressed in MC1061 (wt ffh) and MC1061 (ffh77) cells using the same conditions, PhoA activity in the ffh77 cells (1120 units) was found to be only 39% of the level expressed in wt ffh cells (2860 units) indicating that pVIII-CBD-mediated PhoA export utilizes the co-translational SRP pathway.

### The Membrane-Targeting Peptide

The “membrane-targeting peptide” as used herein refers to a protein, polypeptide, peptide, segment or domain that facilitates the targeting of a recombinant protein to the inner membrane for integration or membrane translocation.

A membrane-targeting peptide may be described according to its topology in the membrane. For example, the term “N-out” referring to a topology of either a prokaryotic or eukaryotic protein means that the N-terminus of the protein is external to or externally protrudes from the membrane containing the protein. (N-out location in bacteria is the periplasmic space.)

The membrane-targeting peptides are recognized by signal recognition particles in a co-translational process. This avoids the release of aggregation-prone hydrophobic segments into the cytoplasm. The membrane-targeting peptide utilized here was selected from naturally or synthetic proteins according to its hydrophobicity as defined below. The hydrophobicity is here calculated by the GES model (Engelman et al. Annu. Rev. Biophys. Chem. 15:321-353 (1986)). This calculation may be performed using the TopPred algorithm (http://mobyle.pasteur.fr/cgibin/MobylePortal/portal.py?form=top pred) (Claros et al. CABIOS 10:685-686 (1994)). It was found that a membrane-targeting peptide could be characterized by a 21 amino acid window in which there is a hydrophobic core sequence of at least 9 amino acids lacking Asp, Glu, Arg, and Lys. A GES hydrophobicity score was assigned to the membrane-targeting peptide using this window of 21 amino acids. The GES score is preferably greater than 1.521 (with a full window size of 21). The term “window” as used herein refers to a continuous stretch of amino acids that can be identified by a computer algorithm programmed to search for a sequence of a specified length.

Examples of such membrane-targeting peptides are pVIII, or mutants of subunit C of F1Fo ATP synthase (F₀c) which are defective in oligomerization (Kol et al. J. Biol. Chem. 281:29762-29768 (2006)) or Pf3 coat protein (Thiaudière et al. Biochemistry 32(45):12186-96 (1993)). These proteins are dependent on YidC for membrane assembly and form trans-membrane segments (Chen et al. J Biol Chem 277(10):7670-5 (2002); Samuelson et al. J Biol Chem. 276(37):34847-52 (2001)).

pVIII protein is the major coat protein of M13 bacteriophage expressed by gene VIII (gVIII). pVIII has evolved to insert efficiently into the inner membrane of E. coli as part of the M13 phage biogenesis process. The precursor form of pVIII (Procoat) (Thiaudière et al. Biochemistry 32(45):12186-96 (1993)) is 73 amino acids while the mature coat protein is 50 amino acids. The pVIII precursor form is cleaved by bacterial SPase upon insertion into the inner membrane.

The precursor pVIII protein preferentially inserts in the membrane with an N-in, C-in topology. (“In” refers to a cytoplasmic location while “out” for this protein in a gram-negative cell refers to a periplasm location. “Topology” refers to the orientation within a lipid bilayer that constitutes a natural or synthetic membrane.)

The pVIII membrane-targeting peptide or variants or derivatives thereof preferably contain a hydrophobic region (residues 44-64 of the precursor form of pVIII as provided in Thiaudière et al. Biochemistry 32(45):12186-96 (1993)) which has been assigned a GES hydrophobicity score of 1.801, (significantly above the 1.52 threshold value for the SRP-dependent DsbA signal sequence). Hence, the pVIII membrane-targeting peptide is recognized by the E. coli signal recognition particle facilitating the membrane integration of a fused membrane protein of interest or to aid the membrane translocation of soluble proteins.

Embodiments of the invention preferably use heterologous membrane proteins of interest with an N-out topology. In such cases, the C-terminus of the N-terminal protein vehicle is preferably extra-cytoplasmic. Accordingly, a series of membrane-targeting constructs were designed and created using standard techniques where pVIII is extended using a segment of bacterial SPase protein (see Examples 2-4, FIGS. 2, 3, 5). This segment preferably includes part of the SPase cytoplasmic loop and a second TM2 plus a plurality of amino acids that extend into the periplasm. Demonstration of SPase TM2 as an efficient export signal was confirmed in Example 5 upon expression of the pVIII-CBD-PhoA9 fusion protein where the PhoA domain was active after translocation across the inner membrane was mediated by SPase TM2.

The C-out modified pVIII membrane-targeting peptide was named pVIII-TM2 (FIG. 2). After SPase cleavage, the mature form of pVIII maintained its position in the membrane with an N-out, C-in topology.

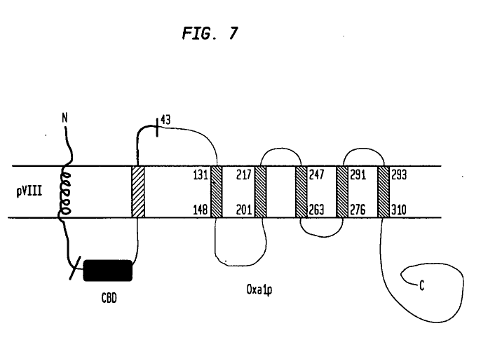

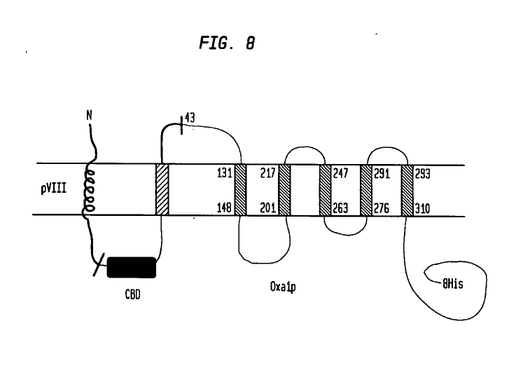

When the membrane protein of interest was fused to the C-terminal end of the trans-membrane segment projecting into the periplasm, the membrane protein of interest was referred to as an N-out membrane protein. “N-out” proteins of interest may be derived from “N-in” proteins of interest by mutagenesis of the open-reading frame (ORF) to create a mutant recombinant gene for fusion to the ORF of the N-terminal protein vehicle for the purpose of expressing a recombinant fusion protein. For example, the recombinant fusion protein ORF may be constructed so that the native signal peptide of the protein of interest is replaced by an ORF encoding one of the membrane-targeting peptides described herein. In embodiments of the invention, methods are provided for producing N-out eukaryotic membrane proteins with correct topology by expressing such proteins as fusions where the fusion junction is located in the E. coli periplasm (see for example, Examples 6-9, FIGS. 7-10).

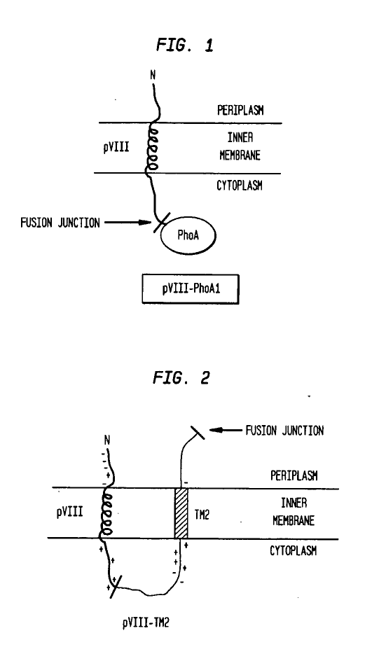

This is in contrast with proteins or domains fused directly to the C-terminus of a membrane-targeting peptide such as pVIII (FIG. 1). In these circumstances, the fusion junction is in the cytoplasm upon membrane assembly of the fusion protein. For example, pVIII-PhoA1 (pVIII bacterial alkaline phosphatase 1) was constructed by ligation of a PhoA reporter domain gene sequence into the EcoRI site downstream of the pVIII ORF. Expression of fusion protein pVIII-PhoA1 (FIG. 1) is robust in bacterial host cells and can be detected by using PhoA monoclonal antibody (Sigma, St. Louis, Mo.). It was found that PhoA activity in these cells is negligible demonstrating that the PhoA domain is cytoplasmic as expected. PhoA is functionally active only when the necessary disulfide bonds are formed by the Dsb system upon export to the periplasm.

An N-terminal protein vehicle for delivering a recombinant eukaryotic membrane protein of interest to a host bacterial inner membrane may include a YidC-dependent ATP synthase subunit C mutant (F₀c L31F) which has two trans-membrane segments where the C-terminal end projects into the periplasm. The C-terminal end can be fused to a target membrane protein of interest. This is optionally fused to a tag at the C-terminal end. Affinity tags may be inserted into the cytoplasmic region between the two trans-membrane segments of the subunit C mutants (FIG. 13).

### The Cytoplasmic Affinity-Binding Domain

Suitable cytoplasmic protein-binding domains within the N-terminal fusion partner include: carbohydrate-binding domains such as chitin-binding domain, cellulose-binding domain and maltose-binding domain; poly-histidine tags, Strep tags and any other protein tags amenable to cytoplasmic expression.

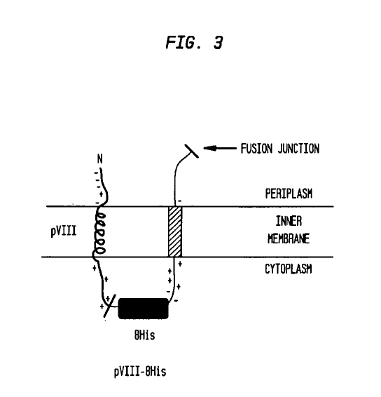

We have discovered that adding an affinity tag to the cytoplasmic loop of the N-terminal fusion partner does not interfere with membrane assembly (FIGS. 3-6, Examples 5-9).

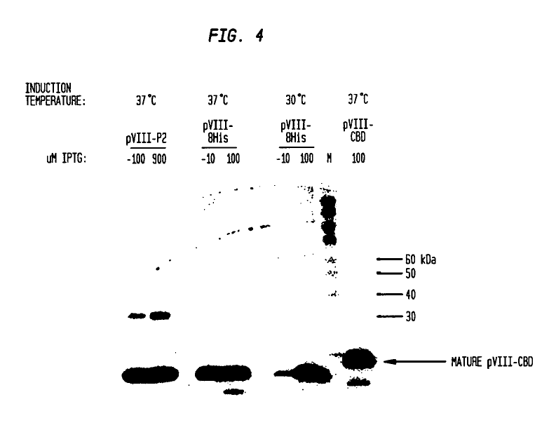

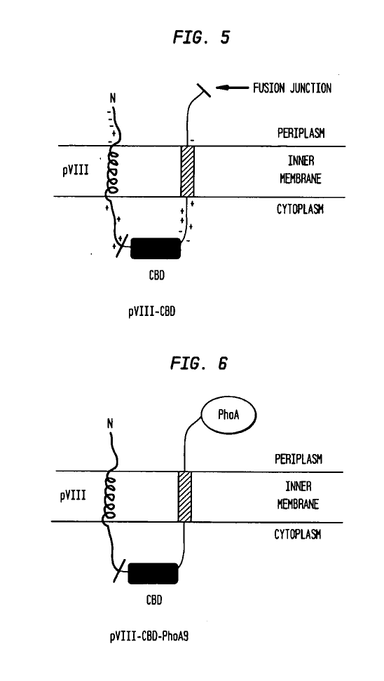

For example, part of the SPase-cytoplasmic loop within pVIII-TM2 was replaced by any affinity tag or detection domain such as a CBD (FIG. 5) or His tag (FIG. 3). In FIG. 3, pVIII-TM2 was mutated to encode a stretch of eight histidines in the cytoplasmic loop at positions 35-42 of SPase. Efficient membrane assembly of pVIII-8His and pVIII-CBD was confirmed by an immunoblot procedure using monoclonal antibody, which preferentially recognizes the mature N-terminus of pVIII after membrane insertion and processing by SPase in vivo (FIG. 4).

In an additional example, pVIII-CBD was shown to mediate the translocation of the PhoA9 protein into the periplasm of E. coli. Expression of fusion protein pVIII-PhoA9 (FIG. 6) is robust in bacterial host cells and can be detected by using PhoA monoclonal antibody or measured by performing phosphatase assays (FIG. 12) as the periplasmic PhoA domain is activated by disulfide bond formation.

### The Trans-Membrane Domain

“TM2” is a generic term for any trans-membrane segment of defined topology. The segment preferably contains for an amino acid sequence window of 21 amino acids a core sequence of at least about 9 amino acids lacking Asp, Glu, Arg, and Lys, and the 21 amino acid window is flanked on the N-terminal end by an amino acid sequence that comprises at least 9 about amino acids having an overall charge of at least +1. The N-terminal end of the trans-membrane domain is preferably located in the cytoplasm and linked to the cytoplasmic affinity-binding domain. The C-terminal end of the trans-membrane domain may under certain circumstances have a net negative charge. The C-terminal end of the trans-membrane region may be linked via an amino acid linker sequence to the protein of interest. The linking amino acid sequence may contain cleavage sites for SPases and/or a heterologous protease. The organization of the linker and cleavage sites are shown in FIGS. 26 and 27. In one example, E. coli SPase recognition sequences were used. E. coli SPase recognition is dependent on TM segment positioning within the inner membrane and also dependent on a relaxed amino acid consensus sequence at −3 and −1 with respect to the point of cleavage (Nielsen et al. Protein Eng. 10(1):1-6 (1997)). Alanine is found most often at −3 and −1 but could be substituted by other small neutral side chains. When proline is present at +1, SPase activity may be inhibited (Barkocy-Gallagher et al. J Biol Chem. 267(2):1231-8 (1992)). However, any amino acid at +1 would be acceptable (except proline) according to in vivo studies on SPase function (Dalbey et al. Protein Sci. 6(6):1129-38 (1997)).

If an intervening TM2 sequence is incorporated between pVIII and the protein of interest, then the N-terminal region of the protein of interest will be translocated into the periplasmic space (FIG. 2).

### Cleavage Sites

In an embodiment of the invention, a cleavage site or sites have been engineered into the fusion protein. Engineered protease cleavage sites are here shown not to interfere with membrane assembly. Examples demonstrating this include: a fusion protein with a cleavage site engineered at the cytoplasmic C-terminal end of pVIII; and a site (Ek, Tev and/or SPase) at the C-terminal end of a second TM2 projecting into the periplasm. Hence, this enables the release of the N-terminus of the membrane protein of interest (the targeted membrane protein) from the membrane-targeting peptide. Protease digestion of the fusion protein may be accomplished in vivo or in vitro.

### Epitopes

Examples of possible epitopes that may be incorporated for antibody detection of an expressed protein of interest are numerous. For example, anti-CBD monoclonal antibody and anti-CBD serum (NEB, Ipswich Mass.) may be used to detect the incorporated CBD. Antibody products are commercially available for poly-His detection. Recognition of the extreme N-terminus of the mature pVIII fusion protein is possible with pVIII monoclonal antibody B62-FE2 (Progen Biotechnik GmbH, Heidelberg Germany) to assess expression of full-length protein (Kneissel et al. J. Mol. Biol. 288: 21-28 (1999)). A suitable extracellular epitope is FLAG which may be recognized by anti-FLAG antibody (Sigma, St. Louis, Mo.).

### The N-Terminal Protein Vehicle

The effectiveness of the pVIII-CBD fusion partner for expression of membrane proteins can be established by an assay in a bacterial host in which endogenous YidC expression is repressed and the viability is compromised in the absence of a complementing protein (van Bloois et al. J. Biol Chem 280: 12996-13003 (2005)). In the assay, the complementing protein was here provided by Oxa1p, which is expressed as a fusion to the pVIII-CBD N-terminal protein vehicle. Oxa1p is a eukaryotic membrane protein, which is the functional homolog of YidC and is found within the mitochondrial inner membrane of eukaryotic cells. Only if the Oxa1p fusion is expressed and inserted correctly in the bacterial inner membrane will the bacteria be viable. FIGS. 7-10 show diagrams of fusion proteins that were expressed and inserted into the E. coli inner membrane in functional form. These results exemplify the utility of the pVIII-CBD fusion partner for heterologous membrane protein expression.

### Fusion Protein Purification

Methods of fusion protein purification using affinity tags to bind to immobilized substrates displayed on resins, beads (including magnetic beads) or matrices known in the art may be employed to purify the expressed membrane proteins.

Isolating the membrane protein of interest may also require removal of pVIII or subunit C mutant membrane-targeting peptide by protease digestion (Enterokinase, for example). Following in vitro digestion of the (pVIII)-(affinity tag)-(protease substrate sequence)-(membrane protein) coupled to a second affinity tag different from the first, the digested fusion protein may be isolated by passing the mixture through a column or using magnetic beads.

Affinity tags in the cytoplasmic loop may be used for fusion protein purification (in constructs lacking a SPase site). In fusion proteins expressed with an SPase site after TM2, the affinity tag in the cytoplasmic loop between the pVIII sequence and TM2 may be used to remove non-processed fusion proteins. For example, in applications where the protein of interest is exported and released by SPase, it is expected that some non-processed full-length fusion protein may be retained within the cytoplasm upon saturation of the export pathway and the SecYEG protein translocase. In this situation, the affinity tag within the N-terminal fusion partner (e.g. pVIII-CBD) may be employed for removal of the non-desirable fusion protein from the soluble protein of interest from within a complex cell lysate.

Export of pVIII-CBD-sp fusions is easily monitored by SDS-PAGE analysis as in vivo cleavage by SPase removes the 19 kDa pVIII-CBD N-terminal partner. In contrast, monitoring typical signal peptide cleavage by SDS-PAGE is often problematic due to the small size (e.g. 18-27 amino acids) of native signal peptides. Using E. coli SPase to cleave overexpressed fusion proteins in vivo is novel and eliminates the need for expensive and often problematic in vitro protease processing steps.

### Expression of Proteins

DNA encoding N-terminal protein vehicles such as pVIII-CBD, pVIII-8His, and F₀C variant L31F fused to DNA encoding a protein of interest may be used to express a wide range of eukaryotic and prokaryotic proteins using the system described herein. The Figures illustrate various aspects of embodiments of the invention. FIGS. 1-10 describe the following:

a membrane-targeting peptide that is incorporated into the inner membrane of a bacterial host where the fusion junction between the membrane-targeting peptide and the target membrane protein is located in the cytoplasm (FIG. 1);

a second trans-membrane segment (TM2) from a second protein is fused at its C-terminal end to a membrane protein of interest or a soluble protein of interest where the fusion junction between the C-terminal end of the membrane-targeting peptide and N-terminal end of the protein of interest is located in the periplasm (FIG. 2). An affinity tag exemplified by a CBD or His tag may be incorporated between the first and second TM segments (FIGS. 3, 5, 6, 7, 8, 9 and 10). An additional affinity tag may be fused to the target membrane protein at its C-terminal end (FIGS. 8, 9, 10).

Examples of engineered fusion proteins expressed with membrane-targeting peptides include: pVIII-PhoA1 (cytoplasmic fusion); pVIII-TM2 (TM2 from SPase added to extend C-terminus to periplasm); pVIII-CBD (cytoplasmic CBD); pVIII-CBD-PhoA9 (cytoplasmic CBD, periplasmic PhoA); pVIII-8His (cytoplasmic 8His); pVIII-CBD-Oxa1p; pVIII-CBD-Oxa1p-8His; pVIII-CBD-Ek-Oxa1p-8His; pVIII-CBD-Tev-Oxa1p-8His; wt C-PhoA9; G23D subunit C-PhoA9; L31F subunit C-PhoA9; and L31F subunit C(10His)-PhoA9. These were derived from the expression clone pVIII-P2 (referred to as Procoat-Lep in Samuelson et al. J. Biol. Chem. 276: 34847-34852 (2001)).

There are multiple different methods for making DNA fusions encoding protein expression constructs. A method described here is not intended to be limiting. This method involved a medium copy pMS119 plasmid, which contains an IPTG-inducible Ptac promoter and the lacIq gene (Furste et al. Gene 48: 119-131 (1986)). Any inducible protein expression vector may be used by incorporation of the nucleotide-coding sequence for any of the N-terminal protein vehicles described herein. For example, the method may contain a T7 promoter and the lacI gene for use in one of many host strains expressing the T7 RNA polymerase. These expression constructs encode the M13 gene VIII 5′ untranslated region (position 1250-1300 of the M13 phage genome) to provide an efficient Shine-Delgarno sequence for protein expression in the bacterial host cell.

(SEQ ID NO: 24)
5′-GTGCCTTCGTAGTGGCATTACGTAACCCGTTTAATGGAAACTTCCT
C-3′

The gene VIII Shine-Delgarno sequence is underlined. It should be noted that any 5′ untranslated region with an E. coli Shine-Delgarno sequence may be employed to initiate pVIII fusion or subunit C fusion protein expression in a bacterial host cell.

All references cited herein, as well as U.S. provisional applications Ser. No. 60/965,649 filed Aug. 21, 2007 and Ser. No. 60/965,729 filed Aug. 22, 2007, are incorporated by reference.

### EXAMPLES

The following bacterial strains are used in the present examples. Cloning and propagation of plasmid DNA occurred in NEB 10-beta-competent E. coli (NEB #C3019H, Ipswich Mass.). The genotype of NEB 10-beta strain is fhuA mcrA f80dlacZDM15 DlacX74 endA1 recA1araD139 D(ara,leu)7697 galU galK rpsL nupG D(mrr-hsdRMS-mcrBC).

DHB4 was used for alkaline phosphatase (PhoA) assays (Jander et al. J. Bacteriol. 178: 3049-3058 (1996)). MB68 is DHB4 with a dsbA gene deletion.

JS7131 was used for YidC-complementation assays (Samuelson et al. Nature 406:637-641 (2000)).

MC1061 was used for protein expression (Casabadan and Cohen J. Mol. Biol. 138:179-207 (1980)).

MG1655 was used as a source of genomic DNA for E. coli gene cloning (Blattner et al. Science 277:1453-74 (1997)).

Abbreviations: P2 refers to the soluble P2 domain of E. coli SPase. Signal peptidase is abbreviated as either Lep or SPase.

### Example 1

### Construction of Plasmid pVIII-PhoA1

Plasmid pVIII-PhoA1 (FIG. 14) was constructed from plasmid pVIII-P2. pVIII-P2 was digested with EcoRI to excise the P2 ORF. The remaining vector fragment pVIII-EcoRI (FIG. 15) was treated with Antarctic Phosphatase (NEB, Ipswich Mass.) to prepare for ligation of the PhoA gene fragment. Primers 344-112 and 344-113 were used to amplify the PhoA reporter gene from MG1655 genomic DNA. MfeI cloning sites are underlined:

Primer 344-113
(SEQ ID NO: 25)
5′-CCACCACAATTGGTCCTGTTCTGGAAAACCGGGCTG-3′
Primer 344-114
(SEQ ID NO: 26)
5′-CCACCACAATTGCCGGCAGCGAAAATTCACTGCC-3′.

PCR amplification of the PhoA gene was accomplished with Phusion High Fidelity DNA polymerase (NEB #F-540S, Ipswich Mass.) according to recommended thermocycling conditions. The PhoA gene fragment was digested with MfeI (NEB #R0589S, Ipswich Mass.) and ligated into the EcoRI sites of the pVIII vector. Ligation clones were sequenced to confirm the entire pVIII-PhoA1 ORF. In the pVIII-P2 and the pVIII-PhoA1 protein fusions, pVIII amino acid 73 was mutated from Ser to Arg. In the pVIII-P2 fusion protein the linker region was Arg73-Gly-Ile-Arg (SEQ ID NO:27), which conforms to the Furin protease recognition sequence: Arg-X—X-Arg (SEQ ID NO:28) (NEB #P8077S, Ipswich Mass.). In pVIII-PhoA1 the amino acid linker sequence is Arg73-Gly-Ile-Gly (SEQ ID NO:27) immediately preceding Pro6 of PhoA. This linker may be altered to include a protease recognition site. For example, the linker sequence may include the Arg-X—X-Arg (SEQ ID NO:28) recognition sequence for Furin to enable in vitro removal of the pVIII-targeting peptide from the protein of interest. In principle, any protease recognition sequence may be inserted into the cytoplasmic linker region at a position C-terminal to pVIII to aid in isolating the protein of interest.

E. coli expression of fusion protein pVIII-PhoA1 (FIG. 1) was robust in strain DHB4 (ΔphoA) upon addition of IPTG. Alkaline phosphatase activity was measured using the standard method by analyzing p-nitrophenyl phosphate (PNPP) hydrolysis (Michaelis et al. J. Bacteriol. 154: 366-375 (1983)). The activity reported in these cells was negligible (FIG. 12) indicating that the PhoA domain was expressed within the cytoplasm as expected.

### Example 2

### Construction of Expression Vector pVIII-TM2

Expression vector pVIII-TM2 (FIG. 16) was constructed from plasmid pVIII-P2 as follows: A segment of the E. coli lepB gene (encoding SPase) was amplified from MG1655 genomic DNA using Taq DNA polymerase and primers 342-239 and 342-240. ApoI restriction sites are underlined:

342-239
(SEQ ID NO: 29)
5′-CCACCAGAATTTCGCGTCAGGCAGCGGCGCAGGCGGCT-3′.
342-240
(SEQ ID NO: 30)
5′-CCAGAATTCGAGGATCCTGACGGGATCTGGAACGGTTC-3′.

The lepB gene fragment was digested with ApoI and ligated into the compatible EcoRI sites of the pVIII-EcoRI vector fragment described in Example 1. Ligation clones were analyzed for the proper orientation of the lepB gene fragment by sequencing the plasmid clones with a primer annealing at the Ptac promoter (NEB #s1260s, Ipswich Mass.). Clone c contained the desired ORF where pVIII is extended by a segment of the E. coli SPase protein. This segment (SPase amino acids 34-91) included part of the SPase cytoplasmic loop and the entire second trans-membrane segment (TM2) plus ten amino acids predicted to extend into the periplasm. This amino acid segment was chosen since the membrane topology of SPase TM2 is well documented and SPase TM2 is known to be an efficient export signal for the periplasmic catalytic domain of SPase (von Heijne et al. Proc. Natl. Acad. Sci. USA 85:3363-3366 (1988)). The end of the coding sequence for the lepB gene fragment was modified by designing primer 342-240 to encode a BamHI site and an EcoRI site for creating in-frame genetic fusions to the pVIII-TM2 ORF. The genetic fusion sequence was modeled after the polylinker sequence of pUC19 where the BamHI/EcoRI sites code for the amino acids SGSSNS (SEQ ID NO:31), an ideal flexible amino acid linker sequence. The final step of constructing expression vector pVIII-TM2 was to eliminate the BamHI recognition sequence immediately following the Ptac promoter: Clone c was partially digested with BamHI, filled-in with Klenow fragment and ligated to reclose the vector. The final desired expression vector with a single BamHI site (at the fusion junction) was named pVIII-TM2 (FIG. 16).

### Example 3

### Construction of Expression Vector pVIII-8His

Expression vector pVIII-8HIS (FIG. 17) was constructed from plasmid pVIII-TM2. pVIII-8HIS contains a poly-His sequence within the cytoplasmic loop to enable protein purification or immunodetection. pVIII-TM2 was mutated to encode a stretch of eight histidines in the cytoplasmic loop (replacing amino acids QAAAQAAA (SEQ ID NO:32) equivalent to positions 35-42 of SPase). This expressed membrane-targeting peptide was named pVIII-8His (FIG. 3) and efficient membrane assembly was confirmed by an immunoblot procedure using pVIII monoclonal antibody, which preferentially recognizes the mature N-terminus of pVIII after membrane insertion and processing by SPase in vivo (FIG. 4).

### Example 4

### Construction of Expression Vector pVIII-CBD

An affinity domain was demonstrated to be inserted into the cytoplasmic loop of pVIII-TM2 without interfering with membrane assembly as follows.

pVIII-CBD (FIG. 5) is a membrane-targeting peptide encoded by expression clone pVIII-CBD (FIG. 18) in which CBD is obtained from Bacillus circulans and was inserted within the cytoplasmic loop of pVIII-TM2. Expression vector pVIII-TM2 was amplified by inverse PCR mutagenesis using Phusion™ DNA polymerase (NEB, Ipswich, Mass.) to isolate the vector fragment for ligation to the CBD gene fragment. Primers 342-890 and 342-891 were used to amplify the pVIII-TM2 vector fragment. The CBD ORF was amplified from NEB (Ipswich Mass.) vector pTYB11 (position 5798-5950) using primers 342-888 and 342-889. Blunt ligation of the two fragments in the proper orientation resulted in creating a 51 amino acid insertion of the CBD ORF following the cytoplasmic QAAAQAAA (SEQ ID NO:32) sequence of pVIII-TM2. Experimental results showed that the pVIII-CBD targeting protein was highly over-expressed upon IPTG addition and the majority of the protein was localized to the E. coli membrane fraction. Binding of pVIII-CBD targeting protein to chitin resin (NEB #S6651S, Ipswich Mass.) was confirmed in the presence of 0.1% triton X-100, a typical concentration of detergent used during the purification of membrane proteins.

Primer 342-890

| | |
|---|---|
| 5′-GGCAGCCGCCTGCGCCGCTG-3′. | (SEQ ID NO: 33) |
| Primer 342-891 | |
| 5′-GGGGACTCACTGGATAAAGCAACG-3′. | (SEQ ID NO: 34) |
| Primer 342-888 | |
| 5′-ACGACAAATCCTGGTGTATCCGCTTG-3′. | (SEQ ID NO: 35) |
| Primer 342-889 | |
| 5′-ATGGCCACCTTGAAGCTGCCAC-3′. | (SEQ ID NO: 36) |

### Example 5

### Construction of pVIII-CBD-PhoA9

Expression clone pVIII-CBD-PhoA9 (FIG. 19) was constructed from expression vector pVIII-CBD to evaluate the membrane topology of membrane-targeting peptide, pVIII-CBD. The pVIII-CBD-PhoA9 fusion protein was designed to contain the amino acid sequence YEPFQIPSGSSNC (SEQ ID NO:37) as the periplasmic linker between SPase TM2 and Pro6 of the PhoA reporter domain. The phoA gene fragment was amplified from MG1655 genomic DNA using Phusion™ DNA polymerase (NEB, Ipswich, Mass.) and forward primer 344-112 and reverse primer 344-114 described in Example 1.

Primer 344-112
(SEQ ID NO: 38)
5′-CCACCACAATTGCCCTGTTCTGGAAAACCGGGCTG-3′.
MfeI site is underlined.

The PCR amplified phoA gene fragment was digested with MfeI and ligated into vector pVIII-CBD prepared by EcoRI digestion. A clone encoding the PhoA gene in the proper orientation was designated pVIII-CBD-PhoA9. Fusion protein expression from this clone was robust upon IPTG addition. Alkaline phosphatase activity was measured in DHB4 (ΔphoA) cells after induction of pVIII-CBD-PhoA9 with 1 mM IPTG for 60 minutes at 30° C. In this experiment, the activity was recorded as 2090 units; a 59-fold higher level than for pVIII-PhoA1 expression (FIG. 12). This showed that the pVIII-CBD-PhoA9 fusion protein was integrated into the inner membrane with the correct C-out topology (as displayed in FIG. 6) since the PhoA domain was active.

### Example 6

### Construction of pVIII-CBD-Oxa1p and Demonstration of Oxa1p Membrane Protein Function In Vivo

Expression clone pVIII-CBD-Oxa1p (FIG. 20) was constructed from pVIII-CBD to demonstrate expression of a functional eukaryotic membrane protein in E. coli. Oxa1p is an integral membrane protein, which naturally resides within the mitochondrial inner membrane. Oxa1p functions as a membrane protein insertase and is required for the assembly of integral membrane respiratory complexes (Luirink et al. FEBS Lett. 13:1-5 (2001). The YidC integral membrane protein from E. coli is a homologue of Oxa1p and has a similar function in bacterial membrane protein biogenesis (van der Laan et al. Proc. Natl. Acad. Sci. USA 100:5801-5806 (2003)). van Bloois et al. (J. Biol. Chem. 280:12996-13003 (2005)) demonstrated that a YidC-Oxa1p chimera is able to complement the loss of YidC in E. coli. We created a chimeric fusion between pVIII-CBD and mature Oxa1p (lacking the N-terminal matrix-targeting peptide) to test the membrane-targeting potential of pVIII-CBD. Strain JS7131 was dependent upon arabinose-induced expression of YidC for growth (Samuelson et al. Nature 406:637-641 (2000)). In contrast, providing glucose as the carbon source turned off YidC expression and resulted in loss of viability. Thus, YidC complementation studies were performed by simply plating transformants on LB agar containing glucose to turn off YidC expression and IPTG which induced expression of YidC-complementing proteins.

The oxa1 gene was amplified from S. cerevisiae strain BY4734 (ATCC #200896) using Phusion™ DNA polymerase (NEB, Ipswich, Mass.) and forward primer 354-1110 and reverse primer 354-1098. Reverse primer 354-1098 contained a silent mutation to destroy a naturally-occurring EcoRI site near the end the oxa1 ORF. The pVIII-CBD vector was prepared by inverse PCR amplification using Phusion™ DNA polymerase (NEB, Ipswich, Mass.) and primer 354-1097 and primer 342-240 described in Example 2. Both amplified fragments were digested with EcoRI and ligated to create expression clone pVIII-CBD-Oxa1. This expression clone expressed Oxa1p (beginning at Asn43) as a fusion to targeting protein pVIII-CBD where the fusion junction projected into the periplasm (FIG. 7).

Strain JS7131 was transformed with expression clone pVIII-CBD-Oxa1p and plated on LB agar containing 0.1% glucose. Transformants were obtained only when 10 μM IPTG was included in the LB-glucose plates. Confirmation of complementation was done by streaking transformants from the 10 μM IPTG plates to plates containing either zero or 10 μM IPTG. Again, cell growth was observed on the 10 μM IPTG plates only. This result showed that JS7131 growth required expression of pVIII-CBD-Oxa1p and that Oxa1p functioned as an integral membrane protein when expressed as a fusion to pVIII-CBD.

### Example 7

### Construction of pVIII-CBD-Oxa1p-8His and Demonstration of Oxa1p Membrane Protein Function In Vivo

The Oxa1p ORF present in expression clone pVIII-CBD-Oxa1 was modified to encode a C-terminal eight-histidine sequence to aid in protein purification. This was accomplished using inverse PCR mutagenesis with Phusion™ DNA polymerase (NEB, Ipswich, Mass.) and primers 358-145 and 358-154.

Primer 358-145
(SEQ ID NO: 39)
5′-CACCATCACCATTGAATTCAGCTTGCTGTTTTGGC-3′.
Primer 358-154
(SEQ ID NO: 40)
5′-ATGGTGATGGTGTTTTTTGTTATTAATGAAGTTTGATTTGTGAAC-
3′

The sequence verified expression clone was named pVIII-CBD-Oxa1p-8His (FIG. 21). The expressed protein was tested for in vivo function using the YidC-complementation assay described in Example 6. It was concluded that pVIII-CBD-Oxa1p-8His functioned in vivo by adopting the topology displayed in FIG. 8.

### Example 8

### Construction of pVIII-CBD-Ek-Oxa1p-8His and Demonstration of Oxa1p Membrane Protein Function In Vivo

The fusion protein ORF in expression clone pVIII-CBD-Oxa1p-8His was modified to encode the Ek protease (NEB #P8070S, Ipswich Mass.) recognition sequence immediately preceding the fusion junction to Oxa1p. This was accomplished by digesting the plasmid expression clone with BamHI and ligating a double-stranded insert comprised of the complementary pair of oligonucleotides: 358-155 and 358-204.

Primer 358-155
(SEQ ID NO: 41)
5′-GATCGGACTACAAAGATGACGATGACAAAG-3′.
Primer 355-204
(SEQ ID NO: 42)
5′-GATCCTTTGTCATCGTCATCTTTGTAGTCC-3′.

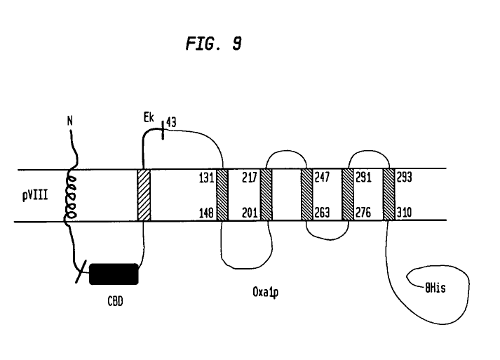

Correct ligation of this oligonucleotide pair resulted in expression clone pVIII-CBD-Ek-Oxa1p-8His (FIG. 22). A unique BamHI restriction site was maintained at the DNA fusion junction to enable cloning of membrane protein genes in place of the oxa1p gene. The “Ek” amino acid sequence inserted at the fusion junction was DYKDDDDKGS (SEQ ID NO:43). The expressed protein was tested for in vivo function using the YidC-complementation assay described in Example 6. pVIII-CBD-Ek-Oxa1p-8His (FIG. 9) was shown to function in vivo. Note that Ek cleaves after the amino acid sequence DDDDK (SEQ ID NO:14). Also, FLAG M2 monoclonal antibody (Sigma F1804, St. Louis, Mo.) specifically recognizes the amino acid sequence DYKDDDDK (SEQ ID NO:13). Thus the inserted “Ek” sequence serves to enable specific immuno-detection of fusion protein and to enable in vivo or in vitro digestion of fusion protein with Enterokinase protease. In vivo digestion of fusion protein requires expressing Enterokinase protease into the E. coli periplasm.

### Example 9

### Construction of pVIII-CBD-Tev-Oxa1p-8His and Demonstration of Oxa1p Membrane Protein Function In Vivo

The fusion protein ORF present in expression clone pVIII-CBD-Oxa1p-8His was modified to encode the Tev protease recognition sequence immediately preceding the fusion junction to Oxa1p. The inserted “Tev” sequence ENLYFQGS (SEQ ID NO:67) enabled in vivo or in vitro digestion of fusion protein with Tev protease. In vivo cleavage of fusion protein required expressing Tev protease into the E. coli periplasm. The pVIII-CBD-Oxa1p-8His expression clone was constructed by digesting clone pVIII-CBD-Oxa1p-8His with BamHI and ligating a double-stranded insert comprised of the complementary pair of oligonucleotides: 358-307 and 358-308.

Primer 358-307

| | |
|---|---|
| 5′-GATCGGAGAACCTGTACTTCCAGG-3′. | (SEQ ID NO: 44) |
| Primer 355-308 | |
| 5′-GATCCCTGGAAGTACAGGTTCTCC-3′. | (SEQ ID NO: 45) |

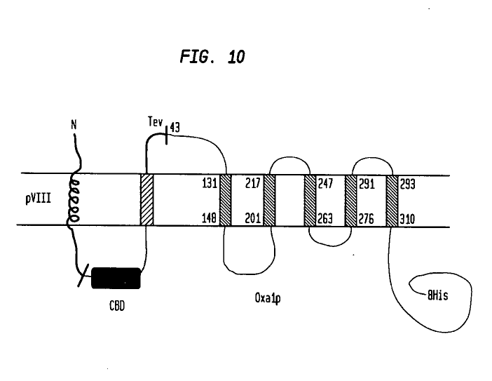

Correct ligation of this oligonucleotide pair resulted in expression clone pVIII-CBD-Tev-Oxa1p-8His (FIG. 23). A unique BamHI restriction site was maintained at the DNA fusion junction to enable cloning of membrane protein genes in place of the oxa1p gene. The expressed protein was tested for in vivo function using the YidC-complementation assay described in Example 6. The results showed that pVIII-CBD-Tev-Oxa1p-8His fusion protein functioned in vivo by adopting the topology displayed in FIG. 10.

### Example 10

### Expression and In Vitro Purification of pVIII-CBD-Ek-Oxa1p-8His Fusion Protein

Expression clone pVIII-CBD-Ek-Oxa1p-8His was transformed into E. coli strain MC1061. A single transformant was cultured in Terrific Broth (Tartof and Hobbs, Bethesda Res. Lab. Focus 9:12 (1987)) plus 100 μg/mL ampicillin at 30° C. until reaching OD600=0.4. The temperature was reduced to 20° C. and protein expression was induced with 100 μM IPTG for 4 hours. Cells were harvested and frozen. The cell membrane fraction was prepared as follows: Cell pellet (1.2 g) from 250 mL culture was resuspended in 5 mL spheroplast buffer [33 mM Tris-HCl (pH 8.0), 20% sucrose]. Lysozyme was added to a final concentration of 0.01 mg/mL and EDTA was added at 1 mM to create spheroplasts by slow stirring at 4° C. for 30 min. Spheroplasts were isolated by centrifugation at 12,000 g for 20 min at 4° C. Spheroplasts were resuspended with 5 mL spheroplast buffer. DNaseI (NEB #M0303S, Ipswich Mass.) was added at 20 units per mL, MgCl₂ was added at 2 mM and protease inhibitor cocktail (Sigma P8849, St. Louis, Mo.) was added before disrupting the spheroplasts with a French press (2 passes at 8,000 psi). This mixture was incubated on ice briefly to allow DNase I to reduce viscosity. Intact cells and any insoluble protein were removed by centrifuging at 24,000 g for 15 min at 4° C. This supernatant was subjected to ultra-centrifugation to pellet the membrane fraction. Ultra-centrifugation was carried out using a Beckman Ti70 rotor at 40,000 rpm (147,000 g) (Beckman-Coulter, Fullerton, Calif.). The membrane pellet was resuspended in 50 mM HEPES-KOH (pH 8.0), 50 mM KCl, 10% glycerol, 0.05% DDM. The membrane fraction was aliquoted into 1004 units and quick-frozen at −80° C.

Protein purification was carried out as follows: 1% DDM was added to a 100 μL aliquot of membrane fraction. The vial was mixed gently for 1 hour at 4° C. to solubilize integral membrane protein. Buffer A [10mM Tris-HCl (pH 8.0), 100 mM KCl, 10% glycerol, 0.1% DDM, 10 mM imidazole] was added to create the Load sample for nickel chelate chromatography. Nickel-NTA agarose resin was pre-washed 3× with buffer A. The load sample (400 μL) was added to 100 μL pre-washed Nickel-NTA resin in an Eppendorf tube and mixed gently for 1 hour at 4° C. The resin void volume was removed and the resin was washed 3× with 200 μL wash buffer [10 mM Tris-HCl (pH 8.0), 100 mM KCl, 10% glycerol, 0.1% DDM, 40 mM imidazole]. Histidine-tagged protein was eluted with two 80 μL volumes of elution buffer [10 mM Tris-HCl (pH 7.0), 100 KCl, 10% glycerol, 0.1% DDM, 400 mM imidazole]. The elution fractions (e1 and e2) as well as other samples taken throughout the purification were analyzed for the presence of pVIII-CBD-Ek-Oxa1p-8His by immunoblot procedures employing anti-pVIII and anti-FLAG monoclonal antibodies (FIG. 11). FIG. 11 shows that pVIII-CBD-Ek-Oxa1p-8His fusion protein may be purified from the membrane fraction of an E. coli transformant using standard procedures. Furthermore, the identical elution profile shown in FIGS. 11A and 11B indicated that the pVIII-CBD membrane-targeting peptide was not subject to in vivo or in vitro proteolytic degradation. This conclusion was made as the FLAG antibody detected the same singular protein species as the pVIII antibody, which specifically recognizes the N-terminus of pVIII (Kneissel et al. J. Mol. Biol. 288: 21-28 (1999)).

### Example 11

### Targeting of PhoA to the Periplasm using ATP Synthase Subunit C Mutant L31F

Three F₀c proteins (wt, G23D and L31F) were tested for membrane-targeting and translocation of the PhoA domain. The tabulated results are shown in FIG. 12. The L31F mutant F₀c-PhoA9 fusion expression level was the same as the wt F₀c-PhoA9 fusion protein expression level. However, the F₀c(L31F)-PhoA9 fusion reported 52% greater alkaline phosphatase activity. This result is explained by the following: 1) the L31F mutant possesses superior characteristics for membrane-targeting and membrane translocation of C-terminally fused proteins and/or; 2) the lack of subunit C-mediated oligomerization allows C-terminally fused proteins to assemble more readily into functional form. Integral membrane proteins may be also be fused to the C-terminus of oligomerization-defective F₀c mutants and targeted into the bacterial membrane in functional form (see hypothetical Example 12). Furthermore, affinity and/or detection epitopes may be incorporated into the cytoplasmic loop of F₀c mutant membrane-targeting peptides. For example, a 10-histidine tag was incorporated into the cytoplasmic loop of F₀c(L31F)-PhoA9 (FIG. 24) to create F₀c(L31F-10His)-PhoA9 (FIG. 25). The 10-histidine tag of F₀c(L31F-10His)-PhoA9 model protein was placed between R41 and Q42 of the F₀c amino acid sequence. The PhoA activity of the F₀c(L31F-10His)-PhoA9 fusion was found to be 80% of the PhoA activity of F₀c(L31F)-PhoA9 (non-His-tagged protein). This showed that affinity tag incorporation did not seriously affect membrane assembly. It was concluded that other affinity tags or detection domains known in the art could be inserted into the cytoplasmic loop of subunit C without affecting membrane assembly of fused proteins.

### Example 12

### Constructs for Expressing a Target Membrane Protein Fused to a Mutant F₀c Membrane-Targeting Peptide

Membrane protein expression clones may be constructed as gene fusions to a mutant F₀c gene, which is known to express an F₀c mutant protein defective in oligomerization. The clones can be constructed according to Examples 2 through 9. The gene encoding the mutant F₀c may replace the gene encoding pVIII-TM2. Consequently, upon expression of the fusion protein, the fusion junction would be present in the periplasm. FIG. 13 shows such an example where F₀c(L31F) is fused to Oxa1p. Preferably, a protease cleavage site would be encoded at the fusion junction as described in Examples 8, 9 and 10. In addition, one or more affinity domains would be incorporated to enable eventual purification of the membrane-targeting peptide after cleavage from the F₀c mutant-targeting protein. The protease cleavage of such fusions may occur in vivo or in vitro.

### Example 13

### Construction of pVIII-CBD-Ek-PhoA18 and pVIII-CBD-sp-Ek-PhoA Vectors

The pVIII-CBD-Ek-PhoA18 expression construct was derived from the pVIII-CBD-PhoA9 construct (FIG. 19) to enable in vivo or in vitro fusion protein cleavage by Enterokinase. The abbreviation “Ek” indicates the inclusion of the FLAG epitope (DYKDDDDK) (SEQ ID NO:13) and corresponding Enterokinase (Ek) protease cleavage site (DDDDK) (SEQ ID NO:14). The PhoA gene insert in each of these constructs may be replaced with a gene of interest encoding either a membrane protein or a soluble protein destined for export to the periplasm. The DNA coding sequence for the Ek/FLAG amino acid sequence was inserted into the unique BamHI site of plasmid pVIII-CBD-PhoA9. Complementary oligonucleotides 358-155 and 358-204 were annealed at a concentration of 1 micromolar in NEB T4 ligase buffer (NEB, Ipswich, Mass.).

358-155 Ek forward oligo:
(SEQ ID NO: 46)
5′P-GATCGGACTACAAAGATGACGATGACAAAG-3′
358-204 Ek reverse oligo:
(SEQ ID NO: 47)
5′P-GATCCTTTGTCATCGTCATCTTTGTAGTCC-3′

where P=phosphorylated nucleotide.

This short oligonucleotide duplex was ligated to plasmid pVIII-CBD-PhoA9 prepared by BamHI digestion and Antarctic phosphatase treatment (NEB #M0289S). Clone pVIII-CBD-Ek-PhoA18 contained the desired DNA insertion that resulted in the following change to the linker region of the expressed fusion protein:
pVIII-CBD-PhoA9 linker: Y₍₁₈₀₎EPFQIPSGS (SEQ ID NO:48) where the “GS” coding region is the cloning site (BamHI) for the gene of interest.
pVIII-CBD-Ek-PhoA18 linker: Y₍₁₈₀₎EPFQIPSGSDYKDDDDKGS (SEQ ID NO:49) where the final “GS” coding region is the cloning site (BamHI) for the gene of interest. The FLAG epitope and Enterokinase recognition site are underlined.

The pVIII-CBD-sp-Ek expression vector was constructed from the pVIII-CBD-Ek vector to enable in vivo fusion protein cleavage by E. coli SPase (FIG. 26C). Accordingly, the designation “sp” indicates that an SPase cleavage site was included at the end of TM2 of fusion constructs to cause constitutive in vivo fusion protein cleavage. pVIII-CBD-sp fusion proteins can be designed to possess any amino acid at +1 except praline following the SPase recognition sequence. The pVIII-CBD-sp-Ek vector was engineered to encode the amino acids SPSAQA (SEQ ID NO:50) at the end of TM2 at positions −6 to −1 relative to the SPase cleavage site. The SPSAQA (SEQ ID NO:50) sequence is a consensus SPase recognition site (Meindl-Beinker et al. EMBO Rep. 7(11):1111-6 (2006)) and Nilsson et al. J Cell Biol. 126(5):1127-32 (1994)); however, other sequences could be processed efficiently. Accordingly, the pVIII-CBD-Ek-TM2 coding sequence for L₍₁₇₃₎IVRSFIYE₍₁₈₁₎ (SEQ ID NO:51) was altered to result in the amino acid sequence L₍₁₇₃₎IVSPSAQÂYE₍₁₈₃₎ (SEQ ID NO:52) and to create the pVIII-CBD-sp-Ek fusion vector where ̂ indicates the SPase cleavage site. The coding sequence was mutated by inverse PCR mutagenesis using the inverse PCR primers 366-361 and 366-362 to amplify the vector with Phusion DNA polymerase (NEB).

366-361 TM2 forward primer:
(SEQ ID NO: 53)
5′P-GCACAGGCGGCATATGAACCGTTCCAGATCCCGTC-3′
366-362 TM2 reverse primer:
(SEQ ID NO: 54)
5′P-AGACGGGGACACAATCAATACGATAGCCAGTAC-3′

After amplification with primers 366-361 and 366-362, the mutated vector was recircularized by ligation to obtain the pVIII-CBD-sp-Ek vector of interest. (See FIG. 27 for a diagram of the spEk linker region.) Fusion protein expression from the pVIIICBDspEk vector resulted in production of the protein of interest with an N-terminal FLAG tag after in vivo processing by E. coli SPase (Example 14, FIGS. 28-29).

Gene fusions (lacking the Ek/FLAG sequence) could be created by amplifying the pVIII-CBD-sp-Ek vector to create a unique EagI restriction enzyme cloning site (Example 15) or a USER™ (NEB, Ipswich, Mass.) compatible single-stranded overhang as described in Examples 17 and 18 below. Such fusion constructs were referred to as pVIII-CBD-sp fusions. EagI gene fusion cloning would result in an AE, AD or AG amino acid pair at +1 and +2 of the protein of interest after SPase cleavage. When the USER™ method (NEB, Ipswich, Mass.) of cloning was applied, seamless pVIII-CBD-sp fusion proteins were produced having any desired sequence at +1 and +2 of the protein of interest (Example 17, FIG. 33).

### Example 14

### Export of Sau3AI Restriction Enzyme to the Periplasm of a Bacterium with an N-Terminal FLAG Tag

Routine expression of the Sau3AI restriction-modification system in the E. coli cytoplasm was problematic due to the toxicity of the Sau3AI endonuclease. The protective Sau3AI methylase modifies the cytosine residues of 5′-GATC-3′ sequences (Seeber et al. Gene 94(1):37-43 (1990)). However, complete Sau3AI protective methylation of E. coli genomic DNA could not be achieved during exponential growth.

To overcome these problems, Sau3AI was exported to the periplasm of an E. coli strain using the methods described herein, but without modification of the genomic DNA by the Sau3AI methylase.

The first attempt at Sau3AI expression employed the pVIII-CBD-sp-Ek vector so that expression and export was monitored by anti-FLAG immunoblotting. FIG. 27 shows the linker region of clone sau4BE. Clone sau4BE expresses a fusion protein designated as pVIII-CBD0sp-Sau3FLAG. After export and SPase cleavage, the N-terminus of this Sau3AI variant was predicted to display the FLAG epitope and the processed protein was predicted to be 58656 daltons. The non-exported and non-processed pVIII-CBD-sp-Sau3FLAG fusion protein was predicted to be 77512 daltons. The sauBE gene (BE=BamHI and EcoRI cloning sites) was amplified from Staphylococcus aureas genomic DNA using primers 370-097 and 368-219 (see below) and ligated into vector pVIII-CBD-sp-Ek digested with BamHI and EcoRI. Note that the BamHI cloning site (GGATCC) corresponds to the Gly and Ser codons following the Ek site (see FIG. 27).

Primer 370-097:
(SEQ ID NO: 55)
5′-CCAGGATCCGAAAGTTATTTGACAAAACAAGCCGTAC-3′
The BamHI restriction site is underlined.
Primer 368-219:
(SEQ ID NO: 56)
5′-CAAGAATTCACAATAAATCTTCAACTTGCTTTTTTATGTAG-3′

The EcoRI restriction site is underlined.

The sauBE recombinant clones were transformed into C2523 (NEB Express) cells (NEB, Ipswich, Mass.). Nine individual clones including sau4BE were grown in LB plus 100 μg/mL ampicillin at 37° C. to OD600=0.4, then induced with 0.1 mM IPTG at 20° C. for 3 hours. After harvest, cells were lysed by 0.1 mg/mL lysozyme treatment and sonicated in sonication buffer A [10 mM Tris-HCl (pH 7.5), 50 mM KCl, 0.1 mM EDTA, 5 mM beta-mercaptoethanol]. Cell lysates were tested for restriction activity on lambda DNA. However, all clones including sau4BE were negative for restriction activity. The same induced cells were analyzed for sauBE4 clone (Sau3FLAG protein) expression by anti-FLAG immunoblot analysis. FIG. 29 shows that a protein of the correct mass (58656 daltons) was expressed upon IPTG induction.

It was concluded that the lack of restriction activity was due to disulfide bonding of the 3 cysteines within Sau3AI upon export to the periplasm by the native Dsb oxidation system which was primarily mediated by the DsbA oxidoreductase enzyme. It is unusual for bacterial cytoplasmic enzymes to possess disulfide bonds so it was reasonable to assume that native Sau3AI did not require disulfide bond(s) for activity. In fact, it was determined that DsbA-mediated disulfide bond formation could result in mis-folded inactive restriction enzyme.

### Example 15

### Expression of Sau3AI Restriction Enzyme to the Periplasm of a dsbA⁻ Strain

Strain MB68 is an E. coli strain lacking the dsbA gene and lacking the periplasmic DsbA oxidoreductase enzyme. pVIII-CBD-sp-Sau3AI clones were constructed without an Ek/FLAG linker for expression in MB68. The sau3AI gene was amplified from Staphylococcus aureus genomic DNA using primers 368-218 and 368-219 and Phusion™ DNA polymerase (NEB, Ipswich, Mass.). The pVIII-CBD-sp-Ek vector was amplified with primers 368-220 and 365-074 with Phusion™ DNA polymerase (NEB, Ipswich, Mass.) to create a unique EagI restriction site at the gene fusion junction.

Sau3AI forward primer 368-218:
(SEQ ID NO: 57)
5′-CAACAACGGCCGAAAGTTATTTGACAAAACAAGCCGTAC-3′.
The EagI site is underlined.
Sau3AI reverse primer 368-219:
(SEQ ID NO: 58)
5′-CAAGAATTCACAATAAATCTTCAACTTGCTTTTTTATGTAG-3′
The EcoRI site is underlined.
spEk vector EagI primer 368-220:
(SEQ ID NO: 59)
5′-CAACAAGCGGCCGCCTGTGCAGACGGGGACACAATC-3′.
The EagI site is underlined.
spEk vector EcoRI primer 365-074:
(SEQ ID NO: 60)
5′-CGGGAATTCAGCTTGGCTGGGC-3′
The EcoRI site is underlined.

After vector/insert ligation, the expressed protein of interest (Sau3Ala) was expected to contain an Alanine in place of the initial Met found in native Sau3AI. As described in Example 13 and shown in FIG. 28, EagI gene fusion cloning resulted in alanine (A) and glutamate (E) amino acids at +1 and +2 of Sau3AI upon cleavage by SPase. DNA sequencing confirmed that clones “sau1” and “sau4” encoded the desired EagI gene fusion. Identical clones containing sau1 and sau4 genes were transformed into MB68 and grown, induced for protein expression and cell lysates were prepared as described in Example 14. Two microliters of cell lysate was incubated with 1 microgram phage T7 genomic DNA for 60 minutes (37° C.) in the presence of 1× NEB buffer 4 (NEB, Ipswich, Mass.).

FIG. 30 shows that the [MB68]sau1 and [MB68]sau4 lysates contain active Sau3AI restriction enzyme when compared to the control digest using 6 units purified BfuCI (NEB #R0636S, NEB, Ipswich, Mass.) which has the same GATC specificity. FIG. 31 demonstrates that the dsbA⁻ strain mutation was preferably required for production of active Sau3AI in the E. coli periplasm as there was no measurable fragmentation of the lambda DNA substrate in lanes 1-8 corresponding to wt DsbA host cells.

### Example 16

### Expression of Periplasmic Sau3AI with an N-Terminal Amino Acid Sequence Identical to Native Sau3AI

Clone saul (Sau3Ala protein) was mutated to create an ATG codon corresponding to the +1 amino acid relative to the SPase cleavage site. The objective of this Example was to produce a Sau3Met protein with same N-terminus as native Sau3AI according the gene sequence reported in Seeber et al. (Gene 94(1):37-43 (1990)). Clone saul was amplified in an inverse PCR reaction with Phusion™ DNA polymerase (NEB, Ipswich, Mass.) using primers 372-913 and 372-915 to create the Ala+1Met mutation:

SauMet forward primer 372-913:
(SEQ ID NO: 61)
5′P-ATGGAAAGTTATTTGACAAAACAAGCC-3′
SauMet reverse primer 372-915:
(SEQ ID NO: 62)
5′P-CGCCTGTGCAGACGGGGACACAATCAATAC-3′

Clones 135D and 145A were sequenced to confirm desired Ala+1Met codon mutation. Then clones 135D and 145A were transformed into MB68 to confirm Sau3AI restriction enzyme expression and activity. The MB68 cultures were grown in LB plus 100 μg/mL ampicillin at 37° C. until OD600=0.4, then shifted to 20° C. for induction with 0.1 mM IPTG for 3 hours. Cell lysates were prepared by 0.1 mg/mL lysozyme treatment followed by sonication in sonication buffer A. FIG. 32 shows that Sau3AI restriction activity was obtained with a Sau3Met (+1) protein expressed in a dsbA⁻ strain.

### Example 17

### Production of Active BfuCI Restriction Enzyme Using a pVIII-CBD-sp Vector Compatible with USER™ Cloning

The BfuCI restriction enzyme from Bacillus fusiformis 1226 had not been over-expressed in E. coli. BfuCI has the same GATC specificity as Sau3AI and was expected to be toxic when expressed in the E. coli for the same reasons as described in Example 14. In this example, BfuCI was expressed from a pVIII-CBD-sp USER™ vector for export to the periplasm. The pVIII-CBD-sp-Ek vector was amplified (without the Ek linker coding region) to create USER™ enzyme generated single-stranded cohesive ends. pVIII-CBD-sp-Ek was amplified with PfuCx polymerase (Stratagene, LaJolla, Calif.) and primers 372-072 and 372-073 and then incubated with the USER™ enzyme (NEB, Ipswich, Mass.) for 30 minutes. The bfucl gene was amplified from Bacillus fusiformis 1226 genomic DNA using PfuCx polymerase and primers 372-074 and 372-075 and then incubated with the USER™ enzyme for 30 minutes.

Primer 372-072:
(SEQ ID NO: 63)
5′-AGGTACC(dU)GAATTCAGCTTGGCTGTTTTGG-3′
Primer 372-073:
(SEQ ID NO: 64)
5′-AGCCTGTGC(dU)GACGGGGACACAATCAATAC-3′
Primer 372-074:
(SEQ ID NO: 65)
5′-AGCACAGGC(dU)GTTTTTGAAACTGAAGAGGCACTT-3′
Primer 372-075:
(SEQ ID NO: 66)
5′-AGGTACC(dU)TATTTGACTTCCTTTACTATTTCTGC-3′

The vector and insert solutions were mixed to enable fragment assembly via 8 and 10 base compatible ends and then directly transformed into C2523 competent E. coli cells. DNA sequencing confirmed that clone BfuC5 encoded the desired BfuCI amino acid sequence (VFETE . . . ) (SEQ ID NO:20) following the SPase cleavage site (FIG. 33). Clone BfuC5 was designed for expression into the periplasm beginning with the +2 position of the native protein. This course of action was taken since the methionine of native BfuCI was expected to be removed by the host cytoplasmic enzyme methionine aminopeptidase when Valine is encoded at position +2 (Meinnel et al. Biochimie. 75(12):1061-75 (1993)). FIG. 33 shows that active BfuCI was expressed from clone BfuC5 in strain MB68. Culture growth, protein expression, lysate preparation, reaction conditions, and presentation of results were the same as described in Example 16.

What is claimed is:

1. A composition, comprising: a recombinant DNA encoding an N-terminal protein vehicle for transporting a protein of interest fused to the vehicle from the cytoplasmic compartment to the envelope of a prokaryotic cell;
the encoded protein vehicle comprising: a membrane-targeting peptide, a cytoplasmic affinity-binding domain, and a trans-membrane segment;
the DNA encoding the protein vehicle being fused to a DNA encoding a protein of interest.

2. A composition according to claim 1, wherein the encoded membrane-targeting peptide is characterized by a Goldman-Engelman-Steitz hydrophobicity score of at least 1.52 for an amino acid window size of 21.

3. A composition according to claim 1, wherein the encoded membrane-targeting peptide has an amino acid window of 21 amino acids such that within the 21 amino acid window there is a hydrophobic core sequence of at least 9 amino acids lacking Asp, Glu, Arg, and Lys.

4. A composition according to claim 1, wherein the encoded membrane-targeting peptide is YidC-dependent.

5. A composition according to claim 4, wherein the encoded membrane-targeting peptide is selected from pVIII, Pf3 coat, and subunit C variant L31F.

6. A composition according to claim 1, wherein the encoded cytoplasmic affinity-binding domain is a carbohydrate-binding domain.

7. A composition, according to claim 6, wherein the encoded cytoplasmic affinity-binding domain is a chitin-binding domain.

8. A composition according to claim 1, wherein the encoded cytoplasmic affinity-binding domain is selected from a His tag and a strep tag.

9. A composition according to claim 1, wherein the encoded trans-membrane segment has an N-terminal and C-terminal end, the segment having a 21 amino acid sequence window such that within the 21 amino acid window there is a core sequence of at least 9 amino acids lacking Asp, Glu, Arg, and Lys and the 21 amino acid window is flanked on the N-terminal end by an amino acid sequence that comprises at least 9 amino acids having an overall charge of at least +1.

10. A composition according to claim 9, wherein the 21 amino acid window is flanked on the C-terminal end by an amino acid linker sequence.

11. A composition according to claim 10, wherein the linker sequence contains a signal peptidase cleavage site.

12. A composition according to claim 10, wherein the linker sequence contains a heterologous protease cleavage site.

13. A composition according to claim 9, wherein the encoded trans-membrane segment is TM2 of signal peptidase.

14. A fusion protein comprising: a membrane-targeting peptide, a cytoplasmic affinity-binding domain, a trans-membrane segment and a protein of interest.

15. A fusion protein according to claim 14, wherein the protein of interest is a toxic protein.

16. A fusion protein according to claim 15, wherein the toxic protein is a restriction endonuclease.

17. A fusion protein according to claim 16, wherein the restriction endonuclease is Sau3AI or isoschizomer.

18. A method of producing a recombinant protein in a gram-negative bacterial host cell, comprising:
(a) obtaining a recombinant DNA characterized in claim 1 fused to a DNA encoding a protein of interest; and
(b) transforming the host cell.

19. A method according to claim 18, wherein the recombinant protein is purified from the host cell by binding the cytoplasmic affinity-binding domain to an affinity substrate.

20. A method according to claim 18, further comprising: assaying the transformed host cell for detection of the protein of interest by binding to an affinity substrate via the cytoplasmic affinity-binding domain.

21. A method according to claim 18, wherein the host cell has a DsbA⁻ phenotype.

22. A method according to claim 21, further comprising: assaying the transformed host cell for determining at least one of enzymatic and binding activity of the protein of interest.

23. A method according to claim 18, wherein the protein of interest is Sau3AI.

24. A method according to claim 18, wherein the protein of interest is BfuCI.

25. A DNA, comprising: nucleotide sequence 631-1986 in SEQ ID NO:23.

26. A method, comprising: producing a restriction endonuclease in the absence of cognate methylation according to claim 18.

27. A method according to claim 26, wherein the restriction endonuclease is Sau3AI or isoschizomer thereof.

28. A vector, comprising: the DNA of claim 1.

29. A host cell, comprising: the vector of claim 28.


SEQUENCE LISTING (67 sequences)

SEQ ID NO: 1 - DNA, 5606 bp, artificial
  completely synthesized plasmid pVIII-phoA1
aagcttgcatgcctgcaggtcgacggatccccggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaattggtcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaataaaaccgcgcccggcagtgaattttcgctgccggcaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 2 - DNA, 4229 bp, artificial
  completely synthesized plasmid pVIII-EcoRI
aagcttgcatgcctgcaggtcgacggatccccggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgctgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 3 - DNA, 4415 bp, artificial
  completely synthesized plasmid pVIII-TM2
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 4 - DNA, 4415 bp, artificial
  completely synthesized plasmid pVIII-8His
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcatcaccatcaccatcaccatcacggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 5 - DNA, 4580 bp, artificial
  completely synthesized plasmid pVIII-CBD
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 6 - DNA, 5957 bp, artificial
  completely synthesized plasmit pVIII-CBD-phoA9
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcctcgaattgccctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaataaaaccgcgcccggcagtgaattttcgctgccggcaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 7 - DNA, 5662 bp, artificial
  completely synthesized plasmid pVIII-CBD-Oxa1p
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcctcgaattcgacgggcccaaatgccaacgatgtctcggaaatccaaacccagttgccttccatcgatgaattaacctcttcagctccttctctttccgcttctacttcggaccttatcgctaacacgacccaaacagtgggcgagttgtcctcccatatagggtacttaaatagcattggcctggcccaaacctggtactggccctcggacattatccaacacgtcttggaggccgttcatgtttactctgggttgccttggtggggaactatcgcggccaccaccatcctcattcgatgcctgatgtttcccctctatgtcaagtcctctgatactgttgctagaaattcccatatcaagcccgagctggacgccttgaataataagctaatgtccactacagatttgcaacaaggtcagctagtcgccatgcaaaggaaaaaactgctctcctcgcacggcattaagaacagatggctggccgcacccatgctacaaattccaatcgcccttgggtttttcaacgcattgagacacatggctaactacccagtagatgggttcgctaatcaaggtgtcgcttggtttacagacttgactcaagcagacccttacttaggtttgcaagtaatcactgccgctgtgttcatctcatttacaaggctggggggtgagactggtgctcaacaattcagttctcccatgaagcgtcttttcactattctaccgatcatttctataccggccacaatgaacttatcgtccgctgtggtcctctactttgcctttaatggtgccttctccgtcctacagacaatgattttgagaaacaaatgggttcgttcgaaactgaagataacagaagtagctaaaccaaggactcctatcgctggcgcttcccccacagagaacatgggcatcttccaatcattaaaacataacattcaaaaggcaagagatcaggcggaaagaaggcaattgatgcaagataatgagaagaagttacaagaaagcttcaaggagaagaggcagaactccaaaatcaaaattgttcacaaatcaaacttcattaataacaaaaaatgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 8 - DNA, 5686 bp, artificial
  completely synthesized plasmid pVIII-CBD-Oxa1p-8His
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcctcgaattcgacgggcccaaatgccaacgatgtctcggaaatccaaacccagttgccttccatcgatgaattaacctcttcagctccttctctttccgcttctacttcggaccttatcgctaacacgacccaaacagtgggcgagttgtcctcccatatagggtacttaaatagcattggcctggcccaaacctggtactggccctcggacattatccaacacgtcttggaggccgttcatgtttactctgggttgccttggtggggaactatcgcggccaccaccatcctcattcgatgcctgatgtttcccctctatgtcaagtcctctgatactgttgctagaaattcccatatcaagcccgagctggacgccttgaataataagctaatgtccactacagatttgcaacaaggtcagctagtcgccatgcaaaggaaaaaactgctctcctcgcacggcattaagaacagatggctggccgcacccatgctacaaattccaatcgcccttgggtttttcaacgcattgagacacatggctaactacccagtagatgggttcgctaatcaaggtgtcgcttggtttacagacttgactcaagcagacccttacttaggtttgcaagtaatcactgccgctgtgttcatctcatttacaaggctggggggtgagactggtgctcaacaattcagttctcccatgaagcgtcttttcactattctaccgatcatttctataccggccacaatgaacttatcgtccgctgtggtcctctactttgcctttaatggtgccttctccgtcctacagacaatgattttgagaaacaaatgggttcgttcgaaactgaagataacagaagtagctaaaccaaggactcctatcgctggcgcttcccccacagagaacatgggcatcttccaatcattaaaacataacattcaaaaggcaagagatcaggcggaaagaaggcaattgatgcaagataatgagaagaagttacaagaaagcttcaaggagaagaggcagaactccaaaatcaaaattgttcacaaatcaaacttcattaataacaaaaaacaccatcaccatcaccatcaccattgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 9 - DNA, 5716 bp, artificial
  completely synthesized plasmid pVIII-CBD-ent-Oxa1p-8His
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcggactacaaagatgacgatgacaaaggatcctcgaattcgacgggcccaaatgccaacgatgtctcggaaatccaaacccagttgccttccatcgatgaattaacctcttcagctccttctctttccgcttctacttcggaccttatcgctaacacgacccaaacagtgggcgagttgtcctcccatatagggtacttaaatagcattggcctggcccaaacctggtactggccctcggacattatccaacacgtcttggaggccgttcatgtttactctgggttgccttggtggggaactatcgcggccaccaccatcctcattcgatgcctgatgtttcccctctatgtcaagtcctctgatactgttgctagaaattcccatatcaagcccgagctggacgccttgaataataagctaatgtccactacagatttgcaacaaggtcagctagtcgccatgcaaaggaaaaaactgctctcctcgcacggcattaagaacagatggctggccgcacccatgctacaaattccaatcgcccttgggtttttcaacgcattgagacacatggctaactacccagtagatgggttcgctaatcaaggtgtcgcttggtttacagacttgactcaagcagacccttacttaggtttgcaagtaatcactgccgctgtgttcatctcatttacaaggctggggggtgagactggtgctcaacaattcagttctcccatgaagcgtcttttcactattctaccgatcatttctataccggccacaatgaacttatcgtccgctgtggtcctctactttgcctttaatggtgccttctccgtcctacagacaatgattttgagaaacaaatgggttcgttcgaaactgaagataacagaagtagctaaaccaaggactcctatcgctggcgcttcccccacagagaacatgggcatcttccaatcattaaaacataacattcaaaaggcaagagatcaggcggaaagaaggcaattgatgcaagataatgagaagaagttacaagaaagcttcaaggagaagaggcagaactccaaaatcaaaattgttcacaaatcaaacttcattaataacaaaaaacaccatcaccatcaccatcaccattgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 10 - DNA, 5710 bp, artificial
  completely synthesized plasmid pVIII-CBD-tev-Oxa1p-8His
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcggagaacctgtacttccagggatcctcgaattcgacgggcccaaatgccaacgatgtctcggaaatccaaacccagttgccttccatcgatgaattaacctcttcagctccttctctttccgcttctacttcggaccttatcgctaacacgacccaaacagtgggcgagttgtcctcccatatagggtacttaaatagcattggcctggcccaaacctggtactggccctcggacattatccaacacgtcttggaggccgttcatgtttactctgggttgccttggtggggaactatcgcggccaccaccatcctcattcgatgcctgatgtttcccctctatgtcaagtcctctgatactgttgctagaaattcccatatcaagcccgagctggacgccttgaataataagctaatgtccactacagatttgcaacaaggtcagctagtcgccatgcaaaggaaaaaactgctctcctcgcacggcattaagaacagatggctggccgcacccatgctacaaattccaatcgcccttgggtttttcaacgcattgagacacatggctaactacccagtagatgggttcgctaatcaaggtgtcgcttggtttacagacttgactcaagcagacccttacttaggtttgcaagtaatcactgccgctgtgttcatctcatttacaaggctggggggtgagactggtgctcaacaattcagttctcccatgaagcgtcttttcactattctaccgatcatttctataccggccacaatgaacttatcgtccgctgtggtcctctactttgcctttaatggtgccttctccgtcctacagacaatgattttgagaaacaaatgggttcgttcgaaactgaagataacagaagtagctaaaccaaggactcctatcgctggcgcttcccccacagagaacatgggcatcttccaatcattaaaacataacattcaaaaggcaagagatcaggcggaaagaaggcaattgatgcaagataatgagaagaagttacaagaaagcttcaaggagaagaggcagaactccaaaatcaaaattgttcacaaatcaaacttcattaataacaaaaaacaccatcaccatcaccatcaccattgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 11 - DNA, 5657 bp, artificial
  completely synthesized plasmid Foc(L31F)-phoA9
aagcttgcatgcctgcaggtcgacggatcgatccccgtgccttcgtagtggcattacgtattttacccgtttaatggaaacttcctcatggaaaacctgaatatggatctgctgtacatggctgccgctgtgatgatgggtctggcggcaatcggtgctgcgatcggtatcggcatcttcgggggtaaattcctggaaggcgcagcgcgtcaacctgatctgattcctctgctgcgtactcagttctttatcgttatgggtctggtggatgctatcccgatgatcgctgtaggtctgggtctgtacgtgatgttcgctgtcgcgtatgaaccgttccagatcccgtcaggatcctcgaattgccctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaataaaaccgcgcccggcagtgaattttcgctgccggcaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 12 - DNA, 5687 bp, artificial
  completely synthesized plasmid Foc(L31F-10His)-PhoA9
aagcttgcatgcctgcaggtcgacggatcgatccccgtgccttcgtagtggcattacgtattttacccgtttaatggaaacttcctcatggaaaacctgaatatggatctgctgtacatggctgccgctgtgatgatgggtctggcggcaatcggtgctgcgatcggtatcggcatcttcgggggtaaattcctggaaggcgcagcgcgtcatcaccatcaccatcaccatcaccatcaccaacctgatctgattcctctgctgcgtactcagttctttatcgttatgggtctggtggatgctatcccgatgatcgctgtaggtctgggtctgtacgtgatgttcgctgtcgcgtatgaaccgttccagatcccgtcaggatcctcgaattgccctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaataaaaccgcgcccggcagtgaattttcgctgccggcaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 13 - protein, 8 aa, artificial
  completely synthetic
DYKDDDDK

SEQ ID NO: 14 - protein, 5 aa, artificial
  completely synthetic
DDDDK

SEQ ID NO: 15 - DNA, 93 bp, artificial
  synthetic
tccccgtctgcacaggcggcatatgaaccgttccagatcccgtcaggatcggactacaaagatgacgatgacaaaggatccgaaagttatttg

SEQ ID NO: 16 - protein, 31 aa, artificial
  synthetic
SPSAQAAYEPFQIPSGSDYKDDDDKGSESYL

SEQ ID NO: 17 - protein, 25 aa, artificial
  synthetic
AYEPFQIPSGSDYKDDDDKGSESYL

SEQ ID NO: 18 - protein, 5 aa, artificial
  synthetic
AFETE

SEQ ID NO: 19 - protein, 5 aa, artificial
  synthetic
MESYL

SEQ ID NO: 20 - protein, 5 aa, artificial
  synthetic
VFETE

SEQ ID NO: 21 - DNA, 5987 bp, artificial
  completely synthetic
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggatcggactacaaagatgacgatgacaaaggatcctcgaattgccctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaataaaaccgcgcccggcagtgaattttcgctgccggcaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 22 - DNA, 5987 bp, artificial; identical to SEQ ID NO: 21
  completely synthetic

SEQ ID NO: 23 - DNA, 5917 bp, artificial
  synthetic
aagcttgcatgcctgcaggtcgacggatcgatccccgtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaaggggaatttcgcgtcaggcagcggcgcaggcggctgccacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaggtggccatggggactcactggataaagcaacgttgaaaaaggttgcgccgaagcctggctggctggaaaccggtgcttctgtttttccggtactggctatcgtattgattgtgtccccgtcagcacaggctgtttttgaaactgaagaggcacttttatataaggcacaagaggcgcgtggtaaaacattcagtgagattgatcagcatggtagattaaatagtaaacgatctacaggtgcgctaggtcagattgttgaagaaagtttttttggatatgaagtgaatagcaatgctgaggctgatttttcgaacttaggtatagagctaaaggtgacgccttttaagcagaataaggataaaagtttgtctgctaaggaacgactagtacttaacattataaactacatgacagaagcggataaaaatttttatgattcgagcttttggaagaaaaatcagaagttacttttgatgttttatgagtggaaaaaagaattaaatcgaggagattataaaattattgaaacacttcttttcgaatatcctgagaaggatttagctgttataaaatctgactgggcattgattcaagggaaaattcgagctgggttggcgcatgagttatcagagggggatacgcaatatttaggggcctgtacgaagggagcaaacaagaattcattacgagaacaacctttttctgatgtacctgcaatgcaacgagctttctctttgaagcaatcttatatgaccgcattagtacggcagtatatatcaaaagaaaagttagtatatttctcaagtcttgaggaactacaggaaaaaacgatagaacaattattaaatgatcgtttcgagccttttatgggaatgacaatggaagaaatggcagaaaagctaaatattcaaattaatcctgggaatcgttctgctgtaccaaatttaattagtgcgttattaggggttaaaggaacaaaactagataaaatagctgaatttgcaaaagcaaatatacaatttaaaacagtgcgtttacaacaaagcggaagacctaaagaaagtatgtcctttaaaaatattgattttaatgaaattattaatgaggaatgggaagatagttatattagaaactattttttagaaacacaaatattatttgttgtctttcaatttgataataacgatttattacgttttaaaggtataaaactttggcatatggctatgaaaactattaataatgagctatttaatttttggaatgaaatacgcagagtattaactgagggggtaattttaacccaatctaaaaagggaatagaaaataacttcccaaaatcaaattttaatggtgtattacatgttcgaccaaaaggggctgatggtagtgataaaatcaaactacctgatggtcaatggattacaaaacagtgttattggttgaatgcaagttatgttgcagaaatagtaaaggaagtcaaataaggtacctgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgcgggtttcgccacctctgacttgagcgtcgatttttgtgatgctcgtcacgggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcgcatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 24 - DNA, 51 bp, M13 phage
  misc_feature (1)..(51): nt 1-51 correspond to 1250-1300 of M13 phage
gtgccttcgtagtggcattacgtattttacccgtttaatggaaacttcctc

SEQ ID NO: 25 - DNA, 36 bp, artificial
  primer
ccaccacaattggtcctgttctggaaaaccgggctg

SEQ ID NO: 26 - DNA, 36 bp, artificial; identical to SEQ ID NO: 25
  primer

SEQ ID NO: 27 - protein, 4 aa, artificial
  completely synthetic
RGIR

SEQ ID NO: 28 - protein, 4 aa, artificial
  completely synthetic
RXXR

SEQ ID NO: 29 - DNA, 38 bp, artificial
  primer
ccaccagaatttcgcgtcaggcagcggcgcaggcggct

SEQ ID NO: 30 - DNA, 38 bp, artificial
  primer
ccagaattcgaggatcctgacgggatctggaacggttc

SEQ ID NO: 31 - protein, 6 aa, artificial
  completely synthetic
SGSSNS

SEQ ID NO: 32 - protein, 8 aa, Escherichia coli
QAAAQAAA

SEQ ID NO: 33 - DNA, 20 bp, artificial
  primer
ggcagccgcctgcgccgctg

SEQ ID NO: 34 - DNA, 24 bp, artificial
  primer
ggggactcactggataaagcaacg

SEQ ID NO: 35 - DNA, 26 bp, artificial
  primer
acgacaaatcctggtgtatccgcttg

SEQ ID NO: 36 - DNA, 22 bp, artificial
  primer
atggccaccttgaagctgccac

SEQ ID NO: 37 - protein, 13 aa, artificial
  completely synthetic
YEPFQIPSGSSNC

SEQ ID NO: 38 - DNA, 35 bp, artificial
  primer
ccaccacaattgccctgttctggaaaaccgggctg

SEQ ID NO: 39 - DNA, 36 bp, artificial
  primer
caccatcaccattgaattcagcttggctgttttggc

SEQ ID NO: 40 - DNA, 45 bp, artificial
  primer
atggtgatggtgttttttgttattaatgaagtttgatttgtgaac

SEQ ID NO: 41 - DNA, 30 bp, artificial
  primer
gatcggactacaaagatgacgatgacaaag

SEQ ID NO: 42 - DNA, 30 bp, artificial
  primer
gatcctttgtcatcgtcatctttgtagtcc

SEQ ID NO: 43 - protein, 10 aa, artificial
  completely synthetic
DYKDDDDKGS

SEQ ID NO: 44 - DNA, 24 bp, artificial
  primer
gatcggagaacctgtacttccagg

SEQ ID NO: 45 - DNA, 24 bp, artificial
  primer
gatccctggaagtacaggttctcc

SEQ ID NO: 46 - DNA, 30 bp, artificial; identical to SEQ ID NO: 41
  oligonucleotide

SEQ ID NO: 47 - DNA, 30 bp, artificial; identical to SEQ ID NO: 42
  oligonucleotide

SEQ ID NO: 48 - protein, 10 aa, artificial
  completely synthetic
YEPFQIPSGS

SEQ ID NO: 49 - protein, 19 aa, artificial
  completely synthetic
EPFQIPSGSDYKDDDDKGS

SEQ ID NO: 50 - protein, 6 aa, artificial
  completely synthetic
SPSAQA

SEQ ID NO: 51 - protein, 9 aa, artificial
  completely synthetic
LIVRSFIYE

SEQ ID NO: 52 - protein, 11 aa, artificial
  completely synthetic
LIVSPSAQAYE

SEQ ID NO: 53 - DNA, 35 bp, artificial
  primer
gcacaggcggcatatgaaccgttccagatcccgtc

SEQ ID NO: 54 - DNA, 33 bp, artificial
  primer
agacggggacacaatcaatacgatagccagtac

SEQ ID NO: 55 - DNA, 37 bp, artificial
  primer
ccaggatccgaaagttatttgacaaaacaagccgtac

SEQ ID NO: 56 - DNA, 41 bp, artificial
  primer
caagaattcacaataaatcttcaacttgcttttttatgtag

SEQ ID NO: 57 - DNA, 38 bp, artificial
  primer
caacaacggccgaaagttatttgacaaaacaagccgta

SEQ ID NO: 58 - DNA, 41 bp, artificial; identical to SEQ ID NO: 56
  primer

SEQ ID NO: 59 - DNA, 36 bp, artificial
  primer
caacaagcggccgcctgtgcagacggggacacaatc

SEQ ID NO: 60 - DNA, 26 bp, artificial
  primer
cgggaattcagcttggctgttttggc

SEQ ID NO: 61 - DNA, 27 bp, artificial
  primer
atggaaagttatttgacaaaacaagcc

SEQ ID NO: 62 - DNA, 30 bp, artificial
  primer
cgcctgtgcagacggggacacaatcaatac

SEQ ID NO: 63 - DNA, 30 bp, artificial
  primer
aggtaccugaattcagcttggctgttttgg

SEQ ID NO: 64 - DNA, 30 bp, artificial
  primer
agcctgtgcugacggggacacaatcaatac

SEQ ID NO: 65 - DNA, 34 bp, artificial
  primer
agcacaggcugtttttgaaactgaagaggcactt

SEQ ID NO: 66 - DNA, 34 bp, artificial
  primer
aggtaccutatttgacttcctttactatttctgc

SEQ ID NO: 67 - protein, 8 aa, Tobacco etch virus
ENLYFQGS